(19) United States
(12) Patent Application Publication
(10) Pub. No.: US 2023/0162860 A1
(43) Pub. Date: May 25, 2023

(54) Methods for Diagnosing and/or Predicting the Risk of Having an Acute Rejection (AR) in a Kidney Transplant Recipient

(71) Applicants: NSERM (Institut National de la Santé et de la Recherche Médicale), Paris (FR); Centre National de la Recherche Scientifique (CNRS), Paris (FR); Université Paris Cité, Paris (FR); Assistance Publique-Hôpitaux de Paris (APHP), Paris (FR)

(72) Inventors: Dany ANGLICHEAU, Paris (FR); Claire TINEL, Paris (FR)

(21) Appl. No.: 17/996,848

(22) PCT Filed: Apr. 21, 2021
(86) PCT No.: PCT/EP2021/060415

§ 371 Date: Oct. 21, 2022

(30) Foreign Application Priority Data
Apr. 22, 2020 (EP) 20305388.9

(51) Int. Cl. G16H 50/20; G01N 33/68; C12Q 1/70; G16H 50/30

(52) U.S. Cl. CPC G16H 50/20; G01N 33/6863; C12Q 1/701; G16H 50/30; G01N 2800/245; G01N 2800/52; G01N 2333/522

(57) ABSTRACT

By using a fully phenotyped cohort of kidney transplant recipients (KTRs), inventors have clearly established the clinical conditions that should be considered when using urinary chemokine levels to noninvasively identify patients at risk of acute rejection (AR). They have developed and validated (in two external validation cohorts) a multiparametric model that predicts individual risk of AR with high accuracy. Accordingly, the invention relates to a method for calculating a probability (p) to have a risk of an acute rejection (AR) in a kidney transplant recipient by using the following equation: (I)

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} . \qquad ( 1 )$$

### FIELD OF THE INVENTION

The invention is in the field of transplantation, particularly, the invention allows to identify whether a subject is at risk of having an acute rejection.

### BACKGROUND OF THE INVENTION

The use of the urinary chemokines C—X—C motif ligand (uCXCL) 9 and uCXCL10 in the daily follow-up of kidney transplant recipients (KTRs) has never seemed so close^{1,2} given the growing evidence of, and interest in, its potential for noninvasive diagnosis and prediction of renal allograft rejection. Positive and negative predictive values (PPVs and NPVs, respectively) varies from 55% to 90%³ suggesting that confounding factors might influence the levels of these proteins and thus impact their diagnostic accuracy and limit their value in clinical practice.

Urinary tract infection (UTI) and BK-virus nephropathy (BKVN)⁴⁻⁶ are two conditions associated with inflammation of the urinary tract and thus with potential increases in the levels of urinary chemokines. However, data on the influence of these confounding factors on uCXCL9 and uCXCL10 are scarce. Moreover, BKV-associated pathology has been described as a progressively damaging infection starting with viruria (a sign of BKV reactivation), followed by BKV viremia (when the immune system fails to control the infection) and ultimately biopsy proven BKVN (when BKV damage can be detected within the allograft)′. To date, very few data exist on the influence of BKV viremia without BKVN on the urinary levels of CXCL9 and CXCL10⁴.

Accordingly, there is a need to identify clinical and biological factors influencing the levels of urinary chemokines and thus provide new tools to diagnose and prevent acute rejection.

### SUMMARY OF THE INVENTION

The invention relates to a method for calculating a probability (p) to have a risk of an acute rejection (AR) in a kidney transplant recipient by using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

In particular, the present invention is defined by the claims.

### DETAILED DESCRIPTION OF THE INVENTION

By using a fully phenotyped cohort of kidney transplant recipients (KTRs), inventors have clearly established the clinical conditions that should be considered when using urinary chemokine levels to noninvasively identify patients at risk of acute rejection (AR). They have developed and validated (in two external validation cohorts) a multiparametric model that predicts individual risk of AR with high accuracy. The major findings of their study are outlined below:
i) Bacteriuria and leukocyturia, hallmarks of urinary tract infection (UTI) are associated with increased urinary levels of CXCL9 and CXCL10;
ii) BK virus viremia, with or without BK-virus nephropathy, is associated with increased urinary levels of CXCL9 and CXCL10;
iii) a simple multiparametric diagnostic model for AR of renal allografts that takes into account all factors influencing urinary chemokine levels is developed;
iv) this model, which includes easily available clinicobiological data and a simple ELISA, achieves unprecedented accuracy for a noninvasive diagnostic tool and is readily translatable to clinical practice.

Every algorithmic method of the present invention is preferably implemented by a computer executing code instructions stored on a memory.

Method for Diagnosing an Acute Rejection (AR)

In a first aspect, the invention relates to a method for calculating a probability (p) to have a risk of an acute rejection (AR) in a kidney transplant recipient by using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

In some embodiments, the invention relates to a computer-implemented method for calculating a probability (p) to have a risk of an acute rejection (AR) in a kidney transplant recipient by using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

In some embodiments, the invention relates to a method for calculating a probability (p) to have a risk of an acute rejection (AR) in a kidney transplant recipient by using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

Wherein:
(x1) is recipient sex,
(x2) is recipient age,
(x3) is estimated glomerular filtration rate (eGFR),
(x4) is donor-specific anti-HLA antibodies score (DSA),
(x5) is blood BKV viral load,
(x6) is urinary tract infection (UTI),
(x7) is CXCL9 and
(x8) is CXCL10.

In some embodiments, the invention relates to a computer-implemented method for calculating a probability (p) to have a risk of an acute rejection (AR) in a kidney transplant recipient by using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

Wherein:
(x1) is recipient sex,
(x2) is recipient age,
(x3) is estimated glomerular filtration rate (eGFR),
(x4) is donor-specific anti-HLA antibodies score (DSA),
(x5) is blood BKV viral load,
(x6) is urinary tract infection (UTI),
(x7) is CXCL9 and
(x8) is CXCL10.

The method according to the invention is suitable for diagnosing an acute rejection (AR) in a kidney transplant recipient by calculating the probability (p).

Typically, in a particular embodiment, the invention relates to a method for diagnosing an acute rejection (AR) in a kidney transplant recipient by calculating a probability (p) of acute rejection for said recipient by using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

In a particular embodiment, the invention relates to a computer-implemented method for diagnosing an acute rejection (AR) in a kidney transplant recipient by calculating a probability (p) of acute rejection for said recipient by using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

In a further embodiment, the invention relates to a method for diagnosing acute rejection (AR) in a kidney transplanted recipient, comprising the following steps:

i) calculating a probability (p) to have a risk of an acute rejection for said recipient using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} ,$$

ii) comparing this probability with a predetermined reference value; and

iii) concluding that the kidney transplant recipient is having or is susceptible to have a risk of an AR when the probability is higher than the predetermined reference value or concluding that the kidney transplant recipient is not having or is not susceptible to have a risk of an AR when the probability is lower than the predetermined reference value.

In a further embodiment, the invention relates to a computer-implemented method for diagnosing acute rejection (AR) in a kidney transplanted recipient, comprising the following steps:

i) calculating a probability (p) to have a risk of an acute rejection for said recipient using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} ,$$

ii) comparing this probability with a predetermined reference value; and

iii) concluding that the kidney transplant recipient is having or is susceptible to have a risk of an AR when the probability is higher than the predetermined reference value or concluding that the kidney transplant recipient is not having or is not susceptible to have a risk of an AR when the probability is lower than the predetermined reference value.

In a further embodiment, the invention relates to a method for diagnosing acute rejection (AR) in a kidney transplanted recipient, comprising the following steps:

i) calculating a probability (p) to have a risk of an acute rejection for said recipient using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} ,$$

ii) comparing this probability with a predetermined reference value; and

iii) concluding that the kidney transplant recipient is having or is susceptible to have a risk of an AR when the probability is higher than the predetermined reference value or concluding that the kidney transplant recipient is not having or is not susceptible to have a risk of an AR when the probability is lower than the predetermined reference value, wherein:
(x1) is recipient sex,
(x2) is recipient age,
(x3) is estimated glomerular filtration rate (eGFR),
(x4) is donor-specific anti-HLA antibodies score (DSA),
(x5) is blood BKV viral load,
(x6) is urinary tract infection (UTI),
(x7) is CXCL9 and
(x8) is CXCL10.

In a further embodiment, the invention relates to a computer-implemented method for diagnosing acute rejection (AR) in a kidney transplanted recipient, comprising the following steps:

i) calculating a probability (p) to have a risk of an acute rejection for said recipient using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} ,$$

ii) comparing this probability with a predetermined reference value; and

iii) concluding that the kidney transplant recipient is having or is susceptible to have a risk of an AR when the probability is higher than the predetermined reference value or concluding that the kidney transplant recipient is not having or is not susceptible to have a risk of an AR when the probability is lower than the predetermined reference value, wherein:
(x1) is recipient sex,
(x2) is recipient age,
(x3) is estimated glomerular filtration rate (eGFR),
(x4) is donor-specific anti-HLA antibodies score (DSA),
(x5) is blood BKV viral load,
(x6) is urinary tract infection (UTI),
(x7) is CXCL9 and
(x8) is CXCL10.

The invention also relates to a computer program product comprising code instructions for implementing any of the above methods for calculating a probability to have a risk of an acute rejection in a kidney transplant recipient, or for diagnosing acute rejection (AR) in a kidney transplanted recipient when it is executed by a computer.

As used herein, the term “probability” refers to whether an event will occur over a specific time period, as in the conversion to relapse, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion. “Risk evaluation,” or “evaluation of risk” in the context of the present invention encompasses making a prediction of the probability, odds, or likelihood that an event or disease state may occur, the rate of occurrence of the event or conversion from one disease state to another, i.e., from a normal condition to relapse or to one at risk of developing relapse. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion to relapse, thus diagnosing and defining the risk spectrum of a category of subjects defined as being at risk of having relapse. In the categorical scenario, the invention can be used to discriminate between normal and other subject cohorts at higher risk of having relapse. In some embodiments, the present invention may be used so as to discriminate those at risk of having relapse from normal, or those having relapse disease from normal.

In the context of the invention, a logistic regression analysis was performed to identify parameters independently associated with AR. Inventors tested for clinically and biologically relevant variables. When several modalities of a single variable were tested (e.g., the presence or absence of DSAs versus the mean fluorescence intensities [MFIs] of the DSAs), the modality associated with the lowest P-value was retained. Several multivariable logistic regression models were then built that included all variables with a P<0.25 in the univariate analysis. Multicollinearity of variables within models was tested by examining the variance inflation factors (VIFs). Values of VIF <2.5 were considered acceptable. A stepwise forward selection and backward elimination procedure was performed to select the best model according to the Akaike Information Criterion (AIC). In a particular embodiment, the analyses were performed with R software (R Development Core Team, version 1.0.44) and GraphPad PRISM® Software (GraphPad Software, San Diego, USA, version 5.02).

In addition to logistic regression, decision trees and neural networks have provided promising preliminary results, by improving diagnostic accuracy. Decision trees are classification models that partition data into subsets based on categories of input variables. This model looks at the data and tries to find the one variable that splits the data into logical groups that are the most different. Gradient Boosting is a variant approach, which resamples the data set several times to generate results that form a weighted average of the resampled data set. Neural networks are capable of modeling extremely complex relationships and are based on pattern recognition and some artificial intelligence processes. They are often used to confirm findings from simple techniques like regression and decision trees. Thus, in some embodiments, decision trees and neural networks can be used in addition to logistic regression.

As used herein term “diagnosing” refers to classifying a disease or a symptom, determining a severity of the disease, monitoring disease progression, forecasting an outcome of a disease and/or prospects of recovery.

As used herein, the term “acute rejection” refers to an acute episode of tissue or transplanted organ injury. Acute rejection characterized by a rejection by the immune system of a tissue transplant recipient when the transplanted tissue is immunologically foreign. More particularly, the acute rejection is characterized by infiltration of the transplant tissue by immune cells of the recipient, which carry out their effector function and destroy the transplant tissue. The onset of acute rejection is rapid and generally occurs in humans within a few weeks after transplant surgery. Generally, acute rejection can be prevented or suppressed with immunosuppressive drugs such as rapamycin, everolimus, cyclosporin, tacrolimus, mycophenolic acid, anti-CD25 monoclonal antibody and lymphocyte-depleting antibodies. Acute rejection episode refers to a single episode of acute rejection which can be recognized and promptly treated, usually preventing organ failure, but recurrent episodes lead to chronic rejection.

In a particular embodiment, the acute rejection includes acute T-cell mediated rejection (TCMR) and borderline rejection, acute antibody-mediated rejection (ABMR), suspected cases of ABMR (when one of the three diagnostic criteria of the Banff classification is lacking) and acute mixed rejection, according to Banff classification system.

As used herein, the term “T-cell mediated rejection” (TCMR) also known as cellular rejection refers to an infiltration of the tissue transplant by T cells and macrophages, intense IFNG and TGFB effects, and epithelial deterioration. Suspicious for TCMR, also called Borderline changes, is characterized by interstitial inflammation, but with insufficient damages to meet the diagnosis of acute TCMR.

As used herein, the term “antibody-mediated rejection, (ABMR)” refers to a type of rejection of transplant tissue or organ by the recipient's immune system. The rejection of a transplanted tissue or organ is triggered by the action of anti-donor's antibodies developed in the recipients against antigens found on the endothelial surface of blood vessels of graft. There are various phenotypes of ABMR: subclinical ABMR, acute ABMR and chronic active ABMR. In the context of the invention, the ABMR is an acute ABMR. In a particular embodiment, it can begin in early stage such as shortly after implantation or at any time during the course of transplant. Acute rejection is often identified clinically by decreased function of the transplanted organ. Lesions of acute active ABMR at the site of the renal transplant characteristically are infiltrated with large numbers of neutrophils, lymphocytes and macrophages in the microvasculature of glomeruli or of peritubular capillaries that cause tissue or organ damage.

As used herein, the term “acute mixed rejection” refers to a rejection in which acute cellular and humoral rejection are involved.

As used herein, the term “transplanted recipient” also called as grafted subject, refers to a subject who has received an organ transplantation. The term “organ transplantation” refers to the procedure of replacing diseased organs, parts of organs, or tissues by healthy organs or tissues. The transplanted organ or tissue can be obtained either from the subject himself (=autograft), from another human donor (=allograft) or from an animal (=xenograft). Transplanted organs may be artificial or natural, whole (such as kidney, heart and liver) or partial (such as heart valves, skin and bone).

As used herein, the term “kidney transplant recipient” refers to a subject who has a kidney transplantation. In a particular embodiment, the kidney transplant recipient refers to any mammals, such as a rodent, a feline, a canine, and a primate. In a particular embodiment, the kidney transplant recipient is a human. In particular, said kidney transplant recipient may further have been grafted with the pancreas, and optionally a piece of duodenum, of the kidney donor.

In a particular embodiment, the kidney transplant recipient is treated with immunosuppressive drugs or other drugs that are currently known in the art or that will be identified in the future.

In a particular embodiment, the kidney transplant recipient is under maintenance immunosuppressive treatment, which means that the subject is administered with one or more immunosuppressive drugs. Immunosuppressive drugs that may be employed in transplantation procedures include but not limited to: azathioprine; tacrolimus; rapamycin derivative such as sirolimus and everolimus; mycophenolic acid such as mycophenolate mofetil and enteric-coated mycophenolate sodium; corticosteroids, and cyclosporin. These drugs may be used in monotherapy or in combination therapies.

In the case of renal transplantation, the following immunosuppressive protocols are usually used. Subjects with primary renal transplantation generally receive an induction treatment consisting of 1 corticoid pulse and 2 injections of basiliximab (Simulect®, a chimeric murine/human monoclonal anti IL2-Rα antibody commercialized by Novartis), in association from day 0 with tacrolimus (Prograf™ or Advagraf™ or Adoport™ or Envarsus™ at 0.1-0.2 mg/kg/day), mycophenolate mofetil (Cellcept from Roche 1-2 g/day; enteric-coated mycophenolate sodium from Novartis 720-1440 mg/day) and corticoseroid, the corticosteroid treatment being progressively decreased until treatment dosage of 10 mg/day, 1 month post-transplantation.

In a further embodiment, the kidney transplant recipient has a secondary or tertiary renal transplantation. In a further embodiment, the kidney transplant recipient is considered as having an increased immunological risk (percentage of anti-T PRA previously peaking above 25% or with preformed donor specific anti-HLA antibodies).

In a particular embodiment, such recipient generally receives 1 corticoid pulse and a short course of depleting antibodies (anti-thymocyte globulin (Thymoglobulin™, Sanofi-Aventis or Grafalon™, Neovii) or alemtuzumab (Campath™, Sanofi-Aventis), 3 to 5 days according to white blood count.

In a further embodiment, said kidney transplant recipient receives from day 0 tacrolimus, mycophenolic acid (mycophenolate mofetil from Roche or enteric-coated mycophenolate sodium from Novartis) and corticoids. The corticoid treatment being progressively decreased of 5 mg every 15 days until treatment dosage of 10 mg/day, 1 month post transplantation.

In a particular embodiment, the method according to the invention, wherein the formula of probability (p) is determined with levels of two proteins expression in a biological sample obtained from a kidney transplant recipient and six clinical parameters of said recipient:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

This algorithmic method is preferably implemented by a computer executing code instructions stored on a memory.

Thus, in a particular embodiment, the method according to the invention is a computer-implemented method wherein the formula of probability (p) is determined with levels of two proteins expression in a biological sample obtained from a kidney transplant recipient and six clinical parameters of said recipient:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

This formula was obtained by studying under R environment and using a multivariable logistic regression to assess the relationship between the outcome “acute rejection” and several predictor variables. Univariate linear regression analysis was performed to determine the clinical and biological parameters that were significantly associated with urinary chemokine levels. The methodology to obtain this formula is fully described in Material and Method section from Tinel et al, (Am J Transplant, 2020). Analyses were performed with R software (R Development Core Team, version 1.0.44) and GraphPad PRISM® Software (GraphPad Software, San Diego, USA, version 5.02).

As used herein, the term β refers to a coefficient for each gene and clinical parameter according to the invention. βi represent the regression β coefficient for each gene and clinical parameter. Typically, the regression β coefficients are determined by the skilled man in the art for each gene using the Bolasso method as described in Erickson, K. F., et al 2016.

As used herein, “β0” refers to intercept. As used herein, the term “intercept” refers to a fixed value used to correct the equation (refers to the interception of the regression curve to the Y axis). Typically, in the context of the invention, β0 is −2.75885 or −3.53296 according to the quantification method.

As used herein, the term “β₁” refers to the coefficient of the sex the kidney recipient subject. In a particular embodiment, the subject is a man. In another embodiment, the subject is a woman.

As used herein, the term “β₂” refers to the coefficient of the age of the subject at the time of biopsy.

As used herein, the term “β₃” refers to the coefficient of eGFR at the time of biopsy.

As used herein, the term “β₄” refers to the coefficient of DSA at the time of biopsy.

As used herein, the term “β₅” refers to the coefficient of BKV load at the time of biopsy.

As used herein, the term “β₆” refers to the coefficient of UTI at the time of biopsy.

As used herein, the term “β₇” refers to the coefficient of CXCL9 at the time of biopsy.

As used herein, the term “β₈” refers to the coefficient of CXCL10 at the time of biopsy.

In a particular embodiment, the method according to the invention, wherein said two proteins are CXCL9 (x7) and CXCL10 (x8).

The coefficients assigned for each parameters may be those described in Table 5 and Table 9.

As used herein, the term “CXCL9” refers to Chemokine (C—X—C motif) ligand 9 and is a small cytokine belonging to the CXC chemokine family that is also known as monokine induced by gamma interferon (MIG). The naturally occurring human CXCL9 has a nucleotide sequence as shown in Genbank Accession number NM_002416 and the naturally occurring human CXCL9 protein has an aminoacid sequence as shown in Genbank Accession number NP_002407. CXCL9 has various role such as induction of chemotaxis, promotion of differentiation and proliferation of leukocytes, and causing tissue extravasation.

As used herein, the term “CXCL10” refers C—X—C motif chemokine 10 (CXCL10) also known as Interferon gamma-induced protein 10 (IP-10) or small-inducible cytokine B10. The naturally occurring human CXCL10 has a nucleotide sequence as shown in Genbank Accession number NM_001565 and the naturally occurring human CXCL10 protein has an aminoacid sequence as shown in Genbank Accession number NP_001556. CXCL10 has various role such as chemoattraction for monocytes/macrophages, T cells, NK cells, and dendritic cells, promotion of T cell adhesion to endothelial cells, antitumor activity, and inhibition of bone marrow colony formation and angiogenesis.

In a particular embodiment, the method according to the invention, wherein the six clinical parameters are: recipient sex (x1), recipient age (x2), estimated glomerular filtration rate (eGFR=x3), donor-specific anti-HLA antibodies score (DSA=x4), blood BKV viral load (x5) and urinary tract infection (UTI=x6).

As used herein, the term the term “recipient sex” also described as x1 refers to the sex of the recipient who receives the kidney organ. Typically, dichotomous categorical variable: gender “M” for masculine or “F” for feminine; gender “M” is taken as a reference (condition with the lowest risk of the event).

As used herein, the term “recipient age” also described as x2 refers to the age of the recipient who receives the kidney organ. Typically, the continuous quantitative variable: recipient age (in years, by each one year) at the time of the biopsy or urine sample.

As used herein, the term “estimated glomerular filtration rate (eGFR)” also described as x3 refers to a measure of the renal function. This test measures the level of creatinine in the blood and uses the result in a formula to calculate a number that reflects how well the kidneys are functioning, said number is called eGFR. In the context of the invention, eGFR is calculated using the MDRD formula (Modification of Diet in Renal Disease) with 4 categories, derived from the CKD classification (Chronic Kidney Disease):
i) category eGFR ≥60 mL/min/1.73 m² is taken as a reference (condition with the lowest risk of the event)
ii) 3 other categories: 30-59 mL/min/1.73 m², 15-29 mL/min/1.73 m², <15 mL/min/1.73 m²

As used herein, the term “donor-specific anti-HLA antibodies score (DSA)” also described as x4 refers to antibodies which are antibody-mediated transplant rejection involving B cell and plasma cell activation. Typically, in the context of the invention, 4 categories are defined according to the MFI (normalized mean fluorescence intensity) with single-antigen flow bead assays on a Luminex platform:
i) beads showing a MFI lower than 500 were considered negative and taken as a reference (condition with the lowest risk of the event)
ii) 3 other categories: 500≤MFI<1000, 1000≤MFI<3000, 3000≤MFI

As used herein, the term “BK virus (BKV)” refers to a human polyomavirus that causes trivial symptoms in the immunocompetent. BKV is ubiquitous in the general population, with infection occurring the first decade of life. After a typically sub-clinical primary infection in early childhood, BKV establishes latency in renal tissues. Reactivation of BKV can result during immunosuppression after AIDS therapy, or the transplant of an organ (particularly kidney) or bone marrow.

As used herein, the term “blood BKV viral load” also described as x5 refers to BKV quantity detected in the blood of a kidney transplanted recipient. Typically, in the context of the invention, blood BKV viral load is expressed as log₁₀ copies/mL. The category BKV viral load <2.4 log is considered negative and taken as a reference. 4 other categories are considered: 1^{st} quartile, 2.4≤x<2.5 log, 2^{nd} quartile, 2.5≤x<3.48 log, 3^{d} quartile, 3.48≤x<4.3 log, upper quartile, ≥4.3 log.

As used herein, the term “urinary tract infection (UTI)” also described as x6 refers to is an infection in any part of urinary system (kidneys, ureters, bladder and urethra). UTI is defined by bacteriuria ≥10³ colony-forming units (CFU) and leukocyturia ≥10⁴ white blood cells per mL; both symptomatic and asymptomatic UTI were included.

As used herein, the term “biological sample” refers to any sample obtained from a transplanted subject, such as a serum sample, a plasma sample, a urine sample, a blood sample, a lymph sample, or a biopsy.

In a particular embodiment, the biological sample is urine sample.

In another embodiment, the urine sample is obtained at any time post transplantation. In one embodiment, the urine sample is obtained at the time of a protocol to determine the probability to have a risk of an AR.

In a particular embodiment, the protein level of CXCL9 and CXCL10 is measured in urine supernatant sample.

Typically, the urine sample is centrifuged at 1000×g for 10 minutes at 4° C. within 4 hours of collection. The supernatants are collected after centrifugation and stored with protease inhibitors at −80° C.

In another embodiment, the RNAm level of CXCL9 and CXCL10 is measured in urine pellet sample.

In a particular embodiment, the expression level corresponds to a group of 2 values corresponding to the expression level of each of the 2 genes (CXCL9 and CXCL10) with further other six values corresponding to the clinical parameters. Typically, the expression level of the 2 genes may be determined by any technology known by a person skilled in the art. In particular, each gene expression level may be measured at the genomic and/or nucleic and/or protein level. In a particular embodiment, the expression level of gene is determined by measuring the amount of nucleic acid transcripts of each gene. In another embodiment, the expression level is determined by measuring the amount of each gene corresponding protein. The amount of nucleic acid transcripts can be measured by any technology known by a man skilled in the art. In particular, the measure may be carried out directly on an extracted messenger RNA (mRNA) sample, or on retrotranscribed complementary DNA (cDNA) prepared from extracted mRNA by technologies well-known in the art. From the mRNA or cDNA sample, the amount of nucleic acid transcripts may be measured using any technology known by a man skilled in the art, including nucleic microarrays, quantitative PCR, microfluidic cards, and hybridization with a labelled probe. In a particular embodiment, the expression level is determined using quantitative PCR. Quantitative, or real-time, PCR is a well-known and easily available technology for those skilled in the art and does not need a precise description. Methods for determining the quantity of mRNA are well known in the art. For example the nucleic acid contained in the biological sample is first extracted according to standard methods, for example using lytic enzymes or chemical solutions or extracted by nucleic-acid-binding resins following the manufacturer's instructions. The extracted mRNA is then detected by hybridization (e. g., Northern blot analysis) and/or amplification (e.g., RT-PCR). Preferably quantitative or semi-quantitative RT-PCR is preferred. Real-time quantitative or semi-quantitative RT-PCR is particularly advantageous.

Other methods of amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e. g. avidin/biotin).

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6× SCC. SCC is a 0.15 M NaCl, 0.015 M Na-citrate).

The nucleic acid primers or probes used in the above amplification and detection method may be assembled as a kit. Such a kit includes consensus primers and molecular probes. A kit also includes the components necessary to determine if amplification has occurred. The kit may also include, for example, PCR buffers and enzymes; positive control sequences, reaction control primers; and instructions for amplifying and detecting the specific sequences.

In a particular embodiment, the method of the invention comprises the steps of providing total RNAs extracted from a biological samples and subjecting the RNAs to amplification and hybridization to specific probes, more particularly by means of a quantitative or semi-quantitative RT-PCR.

In another embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a biological sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

In a particular embodiment, the protein level of CXCL9 and CXCL10 is calculated by concentration of each protein.

In another embodiment, the expression level of the 2 proteins (CXCL9 and CXCL10) can be determined by any technology known in the art consisting but not limited to: ELISA, Ella® (automated microfluidic immunoassay, ProteinSimple), Luminex™ technology, high-performance liquid chromatography (HPLC), electrochemiluminescnece.

In a further embodiment, the protein level of CXCL9 and CXCL10 is measured by enzyme-linked immunosorbent assay (ELISA). Typically, frozen aliquots of urine supernatants were thawed at room temperature immediately before the enzyme-linked immunosorbent assay (ELISA). The samples were used without dilution and were tested in replicate analyses. CXCL10 (Human CXCL10/IP10 Quantikine ELISA Kit, Bio-Techne, Minneapolis, USA) was quantified according to the manufacturer's instructions. For CXCL9, we used an optimized ELISA protocol (Human CXCL9/MIG DuoSet, Bio-Techne)2-4. Briefly, ninety-six-well flat-bottom ELISA plates (Costar, Bio-Techne) were coated with 100 μL/well of 6 μg/mL capture antibody overnight at +4° C. The plates were then washed three times with 300 μL/well of wash buffer (WB) and blocked with 300 μL/well of reagent diluent for 90 minutes at room temperature (RT). The plates were then washed as previously described. Urine supernatant (50 μL/well) was added to the plates together with 50 μL of sample diluent (39.99% v/v of reagent diluent, 59.99% v/v of PBS and 0.03% v/v of Tween-20). The plates were incubated for 120 minutes at RT. Sample diluent (100 μL/well) was added for blank controls (negative controls). Pooled urinary supernatants from patients with BKVN and/or acute rejection were used as internal positive controls. The plates were then washed as before, and detection antibody was added at 100 μL/well. After incubation for 120 minutes at RT, the plates were washed again, and 100 μL/well of HRP-conjugated streptavidin was added. The plates were then incubated at RT while protected from light for 20 minutes and washed as before, after which 100 μL/well of substrate solution (54% Reagent A/46% Reagent B) was added. Following color development at RT for 20 minutes with protection from light, the reaction was stopped with 50 μL/well of stop solution. The plates were gently tapped to ensure thorough mixing. Optical density was measured at 450 nm within 30 minutes using a Multiskan FC plate reader (main cohort) or a Multiskan Sky plate reader (validation cohort, Thermo Fisher Scientific, Illkirch, France). The plate reader was set to subtract the reading of a blank control at 570 nm. The optical densities were derived from 4-parameter logistic regression of the standard curve.

In a further embodiment, the protein level of CXCL9 and CXCL10 is measured by automated microfluidic immunoassay. As example, an automated microfluidic immunoassay may be performed with an Ella® platform. The Ella® platform provides several advantages among which time efficiency, low sample consumption and a high degree of automation (Wessels et al., 2019). Thus, Ella® is a promising tool in the clinical context. As described in Van Manen-Brush et al. (2020), Ella® is an automated instrument that utilizes a cartridge for the assay. The cartridge uses microfluidics to measure the antigen concentration. All the experimental immunoassay steps are performed within a single cartridge designed with individual glass nanoreactors (GNRs). The capture antibody is immobilized on the GNRs, whereas the antigen sample and detection antibody all flow from specific inlet channels to the GNRs. The immunoassay is performed by the addition of the sample to the well; placement of cartridge into the Ella® instrument; and sample flow, wash and detection. All incubations are performed in a single step.

In the context of the present invention, ELISA and Ella® have similar accuracy to assess acute rejection (FIG. 12).

As used herein, the term “predetermined reference value” refers to a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the expression level of the selected peptide in a group of reference, one can use algorithmic analysis for the statistic treatment of the expression levels determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably implemented by a computer executing code instructions stored on a memory. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC. SAS, GB STAT VI0.0 (Dynamic Microsystems, Inc. Silver Spring, Md., USA), etc.

In the context of the invention, the analyses were performed with R software (R Development Core Team, version 1.0.44) and GraphPad PRISM® Software (GraphPad Software, San Diego, USA, version 5.02).

In particular embodiment, predetermined reference value can be obtained from a kidney transplant recipient who has not the following issues: he has not any rejection; he as low or non-detectable level of urine CXCL9 and CXCL10 at protein level, he has neither urinary tract infection nor BKV infection.

In a particular embodiment, the predetermined reference value can be obtained from a subject who has not received an allograft or from a stable allograft recipient.

Method for Determining Whether a Renal Biopsy is Required or not in a Kidney Transplant Recipient

Inventors have performed a decision curve analysis, which calculates a clinical “net benefit” for one or more prediction models in comparison to default strategies of performing a biopsy to all or no patients. FIG. 6A shows the net benefit (identifying true positive cases) of the optimized model compared to the clinical model (eGFR, proteinuria, DSAs) according to the threshold probability. The threshold probability varies according to clinicians and patients' preferences and can be better understood if considered as “biopsies performed to find one rejection” (see secondary x-axis). The blue line, corresponding to the optimized model, has the highest benefit across a wide range of reasonable threshold probabilities. FIG. 6B shows the “net benefit” expressed as biopsies avoided (secondary y-axis), corresponding to true negative cases.

Accordingly, in a second aspect, the invention relates to a method for determining whether a renal biopsy is required or not in a kidney transplant recipient.

In a further embodiment, the invention relates to a method for determining whether a renal biopsy is required or not in a kidney transplant recipient by calculating a probability (p) of acute rejection for said recipient by using the following equation:

p=1/1+exp(−(β0+β1×1+β2×2+β3×3+β4×4+β5×5+β6×6+β7×7+β8×8)).

This algorithmic method is preferably implemented by a computer executing code instructions stored on a memory.

Thus, in a further embodiment, the invention relates to a computer-implemented method for determining whether a renal biopsy is required or not in a kidney transplant recipient by calculating a probability (p) of acute rejection for said recipient by using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

In a particular embodiment, when the kidney transplant recipient is diagnosed as having a risk to have an AR according to the probability as described above, the physician will perform a biopsy.

In another embodiment, when the kidney transplant recipient is diagnosed as not having a risk to have an AR according to the probability as described above, the physician will not perform a biopsy.

Accordingly, the present invention allows to avoid unnecessary biopsies and thus to improve the quality of life of kidney transplanted recipient.

Method for Predicting the Subsequent Occurrence of AR, Predicting Kidney Function and/or Graft Loss

In a third aspect, the invention relates to a method for predicting the subsequent occurrence of an acute rejection in a kidney transplanted recipient.

In a particular embodiment, the invention relates to a method for predicting the subsequent occurrence of an acute rejection in a kidney transplant recipient comprising a step of calculating a probability (p) of acute rejection for said recipient by using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

This algorithmic method is preferably implemented by a computer executing code instructions stored on a memory.

Thus, the invention relates to a computer-implemented method for predicting the subsequent occurrence of an acute rejection in a kidney transplant recipient comprising a step of calculating a probability (p) of acute rejection for said recipient by using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

The method is particularly suitable for predicting the duration of the overall survival (OS), progression-free survival (PFS) and/or the disease-free survival (DFS) of the kidney transplanted recipient. Those of skill in the art will recognize that OS survival time is generally based on and expressed as the percentage of people who survive a certain type of kidney transplantation for a specific amount of time. In general, OS rates do not specify whether kidney transplant recipient survivors are still undergoing treatment or if they have become kidney transplant recipient (achieved remission). DSF gives more specific information and is the number of people with a particular kidney transplantation who achieve remission. Also, progression-free survival (PFS) rates (the number of people who still have kidney transplantation, but their disease does not progress) include people who may have had some success with treatment, but the kidney transplantation has not disappeared completely.

In a particular embodiment, when the probability (p) is higher than a predetermined reference value, the physician can conclude that the kidney transplant recipient is at risk to have a subsequent occurrence of an acute rejection and thus he will increase treatment as described above.

In a particular embodiment, when the probability (p) is lower than a predetermined reference value, the physician can conclude that the kidney transplant recipient is not at risk to have a subsequent occurrence of an acute rejection and thus he will maintain or reduce treatment as described above.

In a particular embodiment, the kidney transplant recipient can have at least one AR from post-transplantation. Said recipient can have can have at least one more acute rejection episodes during his life.

In a forth aspect, the invention relates to a method for predicting whether a kidney transplant recipient is at risk of graft loss.

In a further embodiment, the invention relates to a method for predicting whether a kidney transplant recipient is at risk of graft loss comprising a step of calculating the probability (p) of acute rejection for said recipient by using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

This algorithmic method is preferably implemented by a computer executing code instructions stored on a memory.

Thus, the invention relates to a computer-implemented method for predicting whether a kidney transplant recipient is at risk of graft loss comprising a step of: calculating the probability (p) of acute rejection for said recipient by using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

More particularly, the invention relates to a method for predicting whether a kidney transplant recipient is at risk of graft loss comprising steps of:

i) calculating the probability (p) of acute rejection for said recipient by using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}}$$

and

ii) concluding that the subject is at risk of graft loss when the probability is higher than its predetermined reference value or concluding that the subject is not at risk of graft loss when the probability is lower than its predetermined reference value.

As used herein, the term “predicting” means that the subject to be analyzed by the method of the invention is allocated either into the group of kidney transplant recipient who will lose his graft, or into a group of kidney transplant recipient who will not lose his graft. Typically, said risk is elevated as compared to the average risk in a cohort of transplanted subjects. In the context of the invention, the risk of graft loss in a subject shall be predicted. The term “predicting the risk”, as used herein, refers to assessing the probability according to which the patient as referred to herein will lose graft. As will be understood by those skilled in the art, such an assessment is usually not intended to be correct for 100% of the subjects to be investigated.

As used herein, the term “graft loss” also known as graft failure or transplant loss, refers to loss of function in the kidney transplanted organ or tissue.

In a fifth aspect, the invention relates to a method for predicting the survival time of a kidney transplant recipient comprising the steps of:

i) calculating the probability (p) of acute rejection for said recipient by using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} ,$$

ii) comparing the probability (p) calculated at step i) with its predetermined reference value and

iii) concluding that the subject will have a short survival time when the probability (p) is higher than its predetermined reference value or concluding that the subject will have a long survival time when the probability (p) is lower than its predetermined reference value.

This algorithmic method is preferably implemented by a computer executing code instructions stored on a memory.

Thus, the invention relates to a computer-implemented method for predicting the survival time of a kidney transplant recipient comprising the steps of:

i) calculating the probability (p) of acute rejection for said recipient by using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} ,$$

ii) comparing the probability (p) calculated at step i) with its predetermined reference value and

iii) concluding that the subject will have a short survival time when the probability (p) is higher than its predetermined reference value or concluding that the subject will have a long survival time when the probability (p) is lower than its predetermined reference value.

As used herein, the expression “short survival time” indicates that the subject will have a survival time that will be lower than the median (or mean) observed in the general population of subjects suffering from kidney transplantation. When the subject will have a short survival time, it is meant that the subject will have a “poor prognosis”. Inversely, the expression “long survival time” indicates that the subject will have a survival time that will be higher than the median (or mean) observed in the general population of subjects suffering from kidney transplantation. When the subject will have a long survival time, it is meant that the subject will have a “good prognosis”.

Method for Preventing and/or Treating AR or AR Progression

In a sixth aspect, the invention relates to a method for preventing preventing and/or treating AR or progression of AR in a kidney transplanted recipient, comprising the steps of: (i) performing the method for diagnosing AR according to the method as described above and (ii) administering to said recipient a therapeutically effective amount of a compound selected from the group consisting of azathioprine, tacrolimus, rapamycin derivative (sirolimus and everolimus), mycophenolic acid (mycophenolate mofetil and anteric-coated mycophenolate sodium), corticosteroids, and cyclosporins. These drugs may be used in monotherapy or in combination therapies.

In a further embodiment, the invention relates a method for preventing AR or progression of AR in a kidney transplanted recipient, comprising the steps of:

i) calculating the probability (p) of acute rejection for said recipient by using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} ,$$

ii) administering to said recipient a therapeutically effective amount of a compound selected from the group consisting of azathioprine, tacrolimus, rapamycin derivative (sirolimus and everolimus), mycophenolic acid (mycophenolate mofetil and enteric-coated mycophenolate sodium), corticosteroids, and cyclosporin. These drugs may be used in monotherapy or in combination therapies.

In a seventh aspect, the invention relates to an immunosuppressive therapy for use in treating a kidney transplanted recipient, wherein said kidney transplant recipient subject is diagnosed as being at risk of having an AR by the method according to the invention.

In a further embodiment, the invention relates an immunosuppressive therapy for use in treating a kidney transplanted recipient, comprising the step of calculating the probability (p) of acute rejection for said recipient by using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

As used herein, the terms “treating” “or preventing” refers to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “progression of AR” refers to the evolution of host immune system against the graft. Typically, the graft can have renal damage or can continue to increase the rejection until a chronic rejection.

As used herein, the term “immunosuppressive therapy” refers to immunosuppressive treatment, which means that the subject is administered with one or more immunosuppressive drugs. Immunosuppressive drugs that may be employed in transplantation procedures include azathioprine, tacrolimus, rapamycin derivative (sirolimus and everolimus), mycophenolic acid (mycophenolate mofetil and enteric-coated mycophenolate sodium), corticosteroids, and cyclosporin. These drugs may be used in monotherapy or in combination therapies.

In a particular embodiment, the immunosuppressive drugs can be administered to the kidney transplant recipient simultaneously, separately or sequentially.

As used herein the terms “administering” or “administration” refer to the act of injecting or otherwise physically delivering a substance as it exists outside the body (e.g., immunosuppressive drugs) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

By a “therapeutically effective amount” is meant a sufficient amount of immunosuppressive drugs for use in a method for preventing and/or treating an AR in a subject in need thereof at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic 20 adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

In an eighth aspect, the invention relates to a method for adjusting the immunosuppressive treatment administered to a kidney transplant recipient following its transplantation, comprising the steps of: (i) performing the method for diagnosing AR or the method for determining whether a kidney transplant recipient is at risk of acute rejection according to method of the invention, and (ii) adjusting the immunosuppressive treatment.

In a further embodiment, the invention relates a method for adjusting the immunosuppressive treatment administered to a kidney transplant recipient following its transplantation, comprising the steps of:

i) calculating the probability (p) of acute rejection for said recipient by using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} ,$$

and

ii) adjusting the immunosuppressive treatment.

As used herein, the term “adjusting” refers to changes that can be performed with immunosuppressive treatment. Typically, the physician can reduce the doses of the immunosuppressive treatment when he identifies that the kidney transplant recipient is not at risk of acute rejection according to the invention. In contrary, the physician can perform a biopsy and then increase the doses of the immunosuppressive treatment when he identifies with the method of the invention that the kidney transplant recipient is at risk of acute rejection.

A Method for Immunosuppressive Therapy Weaning

In a ninth aspect, the invention relates to a method for identifying a kidney recipient subject under immunosuppressive therapy as a candidate for immunosuppressive therapy weaning or minimization, comprising the steps of:

i) determining whether the subject is at risk of having an AR by the method according to the invention; and

ii) concluding that the kidney recipient subject is eligible to immunosuppressive therapy weaning or minimization when the subject is not at risk to have an AR.

As used herein, the term “immunosuppressive therapy weaning or minimization” refers to the progressive reduction, and optionally eventually the suppression of an immunosuppressive therapy.

In a further embodiment, the invention relates a method for identifying a kidney recipient subject under immunosuppressive therapy as a candidate for immunosuppressive therapy weaning or minimization, comprising the steps of:

i) calculating the probability (p) of acute rejection for said recipient by using the following equation:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} ,$$

and

ii) concluding that the kidney transplant recipient is eligible to immunosuppressive therapy weaning or minimization when the subject is not at risk to have an AR.

Kit

In a tenth aspect, the present invention relates to a kit for performing the method according to the invention, wherein said kit comprises (i) means for determining the expression level of the CXL9 and CXCL10 in a biological sample obtained from said kidney transplant recipient and (ii) means for determining the six clinical parameters.

In a further embodiment, the kit according to the invention comprises detail and instructions to calculate the probability to have a risk of an acute reject:

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

As used herein, the term “a reagent for the determination of an expression level” is meant a reagent which specifically allows for the determination of said expression level, i.e. a reagent specifically intended for the specific determination of the expression level of the genes and/or proteins comprised in the expression profile level. The kit according to the invention comprises generic reagents useful for the determination of the expression level of any gene and/or protein, such as taq polymerase or an amplification buffer. Age and sex can be determined by asking to the patient.

In a particular embodiment, the kit according to the invention is suitable to perform ELISA to determine the protein expression level of CXCL9 and CXCL10.

In a particular embodiment, the kit according to the invention is suitable to perform an automated microfluidic immunoassay to determine the protein expression level of CXCL9 and CXCL10.

In some embodiments, the kit according to the invention may comprise instructions for:

i) determining whether a kidney transplant recipient is at risk or not of AR and/or AR progression;

ii) determining whether a renal biopsy is required or not in a kidney transplant recipient;

iii) predicting whether a kidney transplant recipient is at risk of graft loss;

iv) predicting the survival time of a kidney transplant recipient; or

v) identifying a kidney recipient subject under immunosuppressive therapy as a candidate for immunosuppressive therapy weaning or minimization

The instructions for this purpose may include at least one reference expression profile. In a particular embodiment, at least one reference expression profile is a graft tolerant expression profile. Alternatively, at least one reference expression profile may be a graft non-tolerant expression profile.

In a further embodiment, the reference expression profile is the reference protein expression level of CXCL9 and CXCL10.

Said reference expression protein profile can be obtained from a kidney transplant recipient who has not the following issues: he has not any rejection; he as low or non-detectable level of urine CXCL9 and CXCL10 at protein level, he has neither urinary tract infection nor BKV infection.

The invention will be further illustrated by the following figures and examples. However, these examples and figures should not be interpreted in any way as limiting the scope of the present invention.

### FIGURES

FIG. 1: BKV infection analysis: sample distribution and chemokine levels. (A.) Euler diagram illustrating the sample distribution according to detection of viremia and histological diagnosis of BKVN and classification into three non-overlapping subgroups according to BKV status: no BKV infection, viremia without BKVN and BKVN. (B. and C.) Urinary CXCL9 and CXCL10 levels in the different subgroups of the total population. (D. and E.) Urinary CXCL9 and CXCL10 levels in the different subgroups after exclusion of cases with significant leukocyturia ≥104/mL (including isolated leukocyturia and UTI) and/or acute rejection. The P-values were obtained using the Kruskal-Wallis test followed by the Dunn's multiple comparisons test. Abbreviations: BKVN, BK-virus nephropathy; Cr, urinary creatinine; UTI, urinary tract infection.

FIG. 2: Urinary tract infection analysis: sample distribution and chemokine levels. (A.) Euler diagram illustrating the sample distribution according to the urinalysis results. The samples were classified into three non-overlapping subgroups according to bacteriological status: no UTI, isolated leukocyturia (≥104/mL) and UTI (bacteriuria ≥103 CFU/mL and leukocyturia ≥104/mL). (B. and C.) Urinary CXCL9 and CXCL10 levels in the different subgroups of the total population. (D. and E.) Urinary CXCL9 and CXCL10 levels in the different subgroups in a restricted population after exclusion of cases with BKV viremia (including isolated viremia and BKVN) and/or acute rejection. The P-values were obtained using the Kruskal-Wallis test followed by the Dunn's multiple comparisons test. Abbreviations: BKVN, BK-virus nephropathy; CFU, colony-forming units; Cr, urinary creatinine; UTI, urinary tract infection.

FIG. 3: Construction, discrimination, performance and calibration of a multiparametric chemokine model. (A. and B.) Diagnostic accuracy of usual biological biomarkers used alone or within the 8-parameter chemokine model (“optimized model”). ROC curves (A.) illustrating the diagnostic performance and Box plots (B.) illustrating the distribution of AUCs generated by 1000 bootstrap replicates to compute 95% CIs. The P-values were obtained from AUC comparisons using the DeLong test. (C.) Box plots comparing the values obtained from the multiparametric chemokine model for the different rejection groups. The P-values were obtained from a Mann-Whitney test. (D.) Calibration curve for the multiparametric chemokine model illustrating the correlations between the observed and predicted values. Goodness of fit was tested by Hosmer-Lemeshow statistics (chi-square values and P-values). (E.) ROC curves illustrating the diagnostic performance of the optimized model for acute rejection diagnosis as compared to a clinical model (based on kidney dysfunction [K], proteinuria [P] and DSAs [D]). (F.) Reclassification analysis between the clinical model (K+P+D) and the optimized model (K+P+D+I+B+C), among patients with and without rejection. Blue histograms indicate the appropriate reclassification to a lower risk in the NR group and to a higher risk in the AR group. Gray histograms indicate a wrong upward reclassification in the NR group, or wrong downward reclassification in the AR group. Net reclassification is given as: (% correct−% wrong) in each group. Abbreviations: AR, acute rejection; AUC, area under the curve; CI, confidence interval; DSAs, donor-specific antibodies; eGFR, estimated glomerular filtration rate; MDRD, modification of diet in renal disease; NR, no rejection; ROC, receiver operating characteristic.

FIG. 4: Internal and external validation of the multiparametric chemokine model. (A-C.) The ROC curves for the multiparametric chemokine model from various sensitivity analyses are shown: when considering only the first biopsy of each patient (N=317 samples; Panel A), according to the indication for biopsy (screening biopsy (N=45) and for-cause biopsy (N=228); Panel B), and according to the time elapsed since transplantation (before 12 months; N=233, and after 12 months; N=140); Panel C). (D.) External validation of the model in two independent cohorts. ROC curves illustrating the diagnostic performances of the same 8 parameters in cohort A (monocentric cohort, 147 urine samples) and cohort B (multicentric cohort, 295 urines samples). Abbreviations: AUC, area under the curve. ROC, receiver operating characteristic.

FIG. 5: Accuracy metrics of the multiparametric model. (A.) Sensitivity, specificity and Youden index ([Sen+Spe]−1) are shown according to the model value. NPV, PPV and accuracy (percentage of correctly classified cases: [TN+TP]/[TN+TP+FN+FP]) are shown in Panel (B.) Optimal cut-off is defined by the maximal Youden index. Two other thresholds were arbitrary chosen: −3 (“low-risk threshold”) to optimize sensitivity and NPV or +1 (“high-risk threshold”) to optimize specificity and PPV. (C.) Model prediction (i.e., risk of acute rejection) according to the model value. The blue dotted line illustrates the example of a patient with a model value of 1.45 corresponding to an 81% risk of acute rejection. Abbreviations: AR, acute rejection; FN, false negative; FP, false positive; NPV, negative predictive value; NR, no rejection; PPV, positive predictive value; Sen, sensitivity; Spe, specificity; Th, threshold; TN, true negative; TP, true positive.

FIG. 6: Clinical utility of the multiparametric chemokine model assessed by a decision curve analysis. (A.) Decision curve analysis showing benefit of performing a biopsy in patients at risk for acute rejection. A reasonable range of threshold probability was chosen and plotted on the x-axis. A secondary x-axis was added to illustrate the number of biopsies to perform to find 1 acute rejection case. The y-axis shows net benefit which is defined as benefit−(harm×threshold probability). Net benefit is given by the following equation: (TP/N)−(FP/N)×pt/(1−pt), with TP being true positives (rejection), FP being false positives, N being the total sample size, and pt the threshold probability. Physicians (taking into account patient preferences) may vary in their propensity to perform a biopsy. One can then consider the benefit/harm ratio in performing a biopsy across several individual scenarios. The blue line shows the net benefit (identifying a true positive case) according to the optimized model compared to a clinical model (gray line) including allograft function, proteinuria and DSAs. Dotted lines show the default strategy of performing a biopsy in all patients (red) or none (green). (B.) Decision curve analysis showing the benefit in identifying true negative patients. This net benefit can also be expressed as the number of biopsies avoided per 100 patients (secondary y-axis). Net benefit of the optimized model (blue line) is plotted as well as the clinical model and the 2 default strategies (“biopsy all”, “biopsy none”). Abbreviations: DSAs, donor-specific antibodies.

FIG. 7: Discrimination accuracy of the multiparametric model for the diagnosis of ABMR among DSA-positive patients. ROC curves illustrating the performances of 2 models for the diagnosis of ABMR among DSA-positive KTR: the multiparametric chemokine model (blue line) compared to the DSA score (blue dashed line). The P-value was obtained from AUC comparisons using the DeLong test. Abbreviations: ABMR, antibody-mediated rejection; AUC, area under the curve; DSA, donor-specific antibodies; KTR, kidney transplant recipients; ROC, receiver operating characteristic.

FIG. 8: Sample distribution and chemokine levels in the cross-sectional study. (A.) Euler diagram illustrating the sample distribution according to detection of viruria, BKV-DNAemia and histological diagnosis of BKVN and the constitution of four non-overlapping groups according to BKV status: no BKV infection, BKV viruria, BKV-DNAemia without BKVN and BKVN. (B.) Urine BKV viral load among the different groups (viruria, BKV-DNAemia and BKVN) and its correlation with uCXCL10/cr levels. Similarly, the blood BKV viral load in the BKV-DNAemia and BKVN groups and its correlation with uCXCL10/cr levels is shown. Correlations were computed from Pearson's test. (C.) Urinary CXCL10 levels according to the 4 BKV groups in the total population and in a restricted population after exclusion of confounding factors. In this restricted population, we excluded samples with significant UTI, with no cytobacterial examination available, with a concurrent acute rejection diagnosis or with inadequate biopsy. P-values were obtained from a Kruskal-Wallis test followed by Dunn's multiple comparisons. BKVN: BKV-associated nephropathy; Cr: urinary creatinine; Ln, natural logarithm; UTI, urinary tract infection.

FIG. 9: BKV-DNAemia prognosis analysis. (A.) Variable importance measures from a random forest analysis. A total of 1000 classification trees were built to address the endpoint “50% eGFR decrease” in the 63 patients with BKV-DNAemia (nested case-control study). Fourteen variables were included among the biological and histological data. The mean decrease in Gini is the average of a variable's total decrease in node impurity, weighted by the proportion of samples reaching that node in each individual decision tree. A higher mean decrease in Gini indicates higher variable importance. (B.) Kaplan-Meier curves illustrating survival before the occurrence of a 50% eGFR decrease in the low- and high-CXCL10 groups (upper panel) and in two groups of viremic patients with or without BKVN (lower panel). The P-value was computed from a log-rank test. (C.) Histograms comparing DSA incidence in the low- and high-CXCL10 groups (upper panel) at different time points: preformed DSAs, DSAs at the time of biopsy and post-BKV de novo DSAs. The lower panel illustrates the post-BKV occurrence of acute rejection, TCMR and AMR. The P-value was computed from Fisher's exact test. The low-CXCL10 group was defined by uCXCL10/cr≤12.86 ng/mmol, and the high-CXCL10 group was defined by uCXCL10/cr>12.86 ng/mmol. Abbreviations: AMR, antibody-mediated rejection, BKVN, BKV-associated nephropathy; cr, urinary creatinine; ci, interstitial fibrosis; ct, tubular atrophy; DSAs, donor-specific antibodies; eGFR, estimated glomerular filtration rate; g, glomerulitis; i, interstitial infiltrate; i-IFTA, inflammation within areas of interstitial fibrosis and tubular atrophy; ptc, peritubular capillaritis; t, tubulitis; TCMR, T-cell mediated rejection; ti, total inflammation.

FIG. 10: Tapering of the maintenance immunosuppressive regimen. In the case-control study (63 patients with BKV-DNAemia), evolution of the mycophenolic acid daily dose (A.) and the tacrolimus trough levels (B.) at different time points following BKV-associated biopsy according to the uCXCL10/cr group. The low-CXCL10 group was defined by uCXCL10/cr≤12.86 ng/mmol, and the high-CXCL10 group was defined by uCXCL10/cr>12.86 ng/mmol. In the longitudinal study (60 patients with BKV-DNAemia), evolution of the mycophenolic acid daily dose (C.) and the tacrolimus trough levels (D.) at different time points following the 1st positive BKV viral load and according to the occurrence of subsequent rejection. Data are presented as the mean±SD values. P-values were obtained via the Mann-Whitney test. The proportions of patients on or off each treatment were compared using Fisher's exact test in Panels A-D without any significant differences. Abbreviations: MPA, mycophenolic acid; ns, not significant; SD, standard deviation.

FIG. 11: Longitudinal study of urinary CXCL10 in BKV viremic patients. (A.) Urinary CXCL10/cr and blood BKV viral load trajectory analyses in the longitudinal cohort including 60 single patients with BKV-DNAemia. Trajectories were computed by regression from longitudinal assessments of uCXCL10/cr (samples collected at biopsy and each outpatient clinic visit during the 1st year post-transplantation, black line) and all available blood BKV viral loads over the same period (blue line). Dotted lines indicate the confidence interval of each group. (B.) Urinary CXCL10/cr trajectory according to uCXCL10/cr threshold at first BKV-DNAemia. The low-CXCL10 group (gray line) is defined by uCXCL10/cr≤12.86 ng/mmol, and the high-CXCL10 group is defined by uCXCL10/cr>12.86 ng/mmol (burgundy line). (C.) Kaplan-Meier curves illustrating survival before the occurrence of a 25% eGFR decrease in the low-CXCL10 (gray curve) and high-CXCL10 (burgundy curve) groups. The P-value was computed from a log-rank test. (D.) Patients were divided according to the occurrence of a post-BKV acute rejection episode (black line) or not (gray line). Dotted lines indicate the confidence interval of each group. Abbreviations: cr, urine creatinine; d, days; eGFR, estimated glomerular filtration rate.

FIG. 12: Clinical validation of Ella®-measured urine chemokine for acute rejection assessment. Diagnostic accuracy (C-statistics) of Ella® results tested against reference ELISA results. ROC curves illustrating the diagnostic performance of the 8-parameter chemokine model, when trained on Ella® or ELISA results.

### Example 1

Material & Methods

Population

We retrospectively considered all consecutive adult patients who received a kidney transplant at Necker Hospital (Paris, France) between February 2011 and February 2016 and selected those with (i) adequate allograft biopsies, (ii) concomitant urine samples available for research and cytobacterial examination, and (iii) blood BKV viral load measurements obtained within ten days before or after biopsy.

Extensive protocol for urinary chemokines quantification by enzyme-linked immunosorbent assay (ELISA) is provided in the present invention, as well as details on histology grading of biopsies, and donor-specific antibodies (DSAs), BK viremia and UTI assessments.

The study was approved by the Ethics Committee of Ile-de-France XI (#13016), and all participating patients provided written informed consent.

Statistical Analyses

The results are presented as the mean±standard deviation (SD) for continuous variables except for the time from transplant to biopsy, viral loads and chemokine levels, which are presented as the median and interquartile range (IQR). Frequencies of categorical variables are presented as numbers and percentages. The distribution of each biomarker exhibited considerable positive skewness, which was substantially reduced through natural logarithm transformation. We compared groups using the Mann-Whitney or Kruskal-Wallis tests, followed by Dunn's posttests when appropriate.

Univariate linear regression analysis was performed to determine the clinical and biological parameters that were significantly associated with urinary chemokine levels. To fulfill the linear regression assumptions, we examined the residuals for normality (both graphically and using normality tests), linearity and homoscedasticity.

Logistic regression analysis was performed to identify parameters independently associated with AR. We tested for clinically and biologically relevant variables. When several modalities of a single variable were tested (e.g., the presence or absence of DSAs versus the mean fluorescence intensities [MFIs] of the DSAs), the modality associated with the lowest P-value was retained.

Several multivariable logistic regression models were then built that included all variables with a P<0.25 in the univariate analysis. Multicollinearity of variables within models was tested by examining the variance inflation factors (VIFs). Values of VIF <2.5 were considered acceptable⁸. A stepwise forward selection and backward elimination procedure was performed to select the best model according to the Akaike Information Criterion (AIC). For internal validation, the abilities of the different models to discriminate AR cases from non-AR cases were determined through quantification of the area under the curve (AUC) values for receiver operating characteristic (ROC) curves. We applied a bootstrap resampling procedure with 1000 repetitions to compute the 95% confidence intervals (CIs). Sensitivities, specificities, NPVs and PPVs are given at the optimal thresholds given by the Youden index⁹. The AUC values of the different models (paired ROC curves) were compared by generating an estimated covariance matrix¹⁰. We tested the calibration of the prediction model both graphically and with the Hosmer-Lemeshow test for goodness of fit¹¹. We performed sensitivity analyses including the indication for biopsy, time from transplantation and only the first biopsy for patients who provided multiple samples. For external validation, we tested the reproducibility of the model on two independent cohorts. Next, we assessed the additive value of the optimized model descriptively by the reclassification in the AR (event) and no rejection (NR, no event) groups. The Net Reclassification Index or the Integrated Discrimination Improvement were not assessed because they could have raised concerns in the settings of nested logistic regression models. Finally, a decision curve analysis was performed to assess the clinical utility of the mode^{12,13}.

Analyses were performed with R software (R Development Core Team, version 1.0.44) and GraphPad PRISM® Software (GraphPad Software, San Diego, USA, version 5.02).

External Validation Cohorts

For independent validation, we quantified uCXCL9 and uCXCL10 in urinary specimens collected at the time of biopsy in two external cohorts: a French single-center cohort (A) and a European multicenter cohort (B). The screening process and characteristics of included vs excluded patients/samples of these two validation cohorts is provided in the present invention.

Results

Study Cohort

According to the inclusion criteria, 391 triplets of samples (i.e., allograft biopsy/urine/BKV viremia samples) corresponding to 329 individual patients were collected (data not shown).

Biopsies were performed at a median time of 8 months post-transplantation (Table 1), of which 88.2% were clinically indicated, most frequently (54.7%) for a rise in serum creatinine. At the time of biopsy, the mean estimated glomerular filtration rate (eGFR) was 36.8±15.6 mL/min, and the mean proteinuria-to-creatininuria ratio was 0.8±1.7 g/g. DSAs were detected in 40.6% of cases. BKV viremia was detectable in 15.9% of cases, with a median viral load of 3.5 [2.5-4.3] log 10 copies/mL. Urinalysis showed that 10% of cases exhibited UTI according to currently accepted criteria. The most frequent pathologic diagnosis was interstitial fibrosis and tubular atrophy (IF/TA, 39.4%). AR was found in 24.3% of biopsy specimens (N=95), and 5.9% displayed BKVN (N=23).

Non-alloimmune inflammation increases urinary CXCL9 and CXCL10 levels

We first sought to identify which clinical or biological variables might be associated with increased urinary levels of CXCL9 and CXCL10. Univariate linear regression was performed for 18 variables (Table 2). No donor or recipient demographic variables were significantly associated with urinary chemokine profiles.

Both chemokines were significantly increased in the presence of DSAs (P<0.05) and histological diagnoses of AR (P<0.001) and were also increased in the presence of leukocyturia, UTI, detectable viremia or BKVN (Table 2).

Urinary levels of CXCL9 and CXCL10 are similarly increased in BKV viremia and BKVN

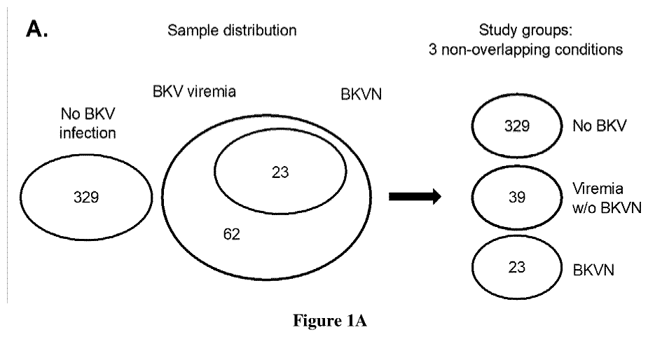

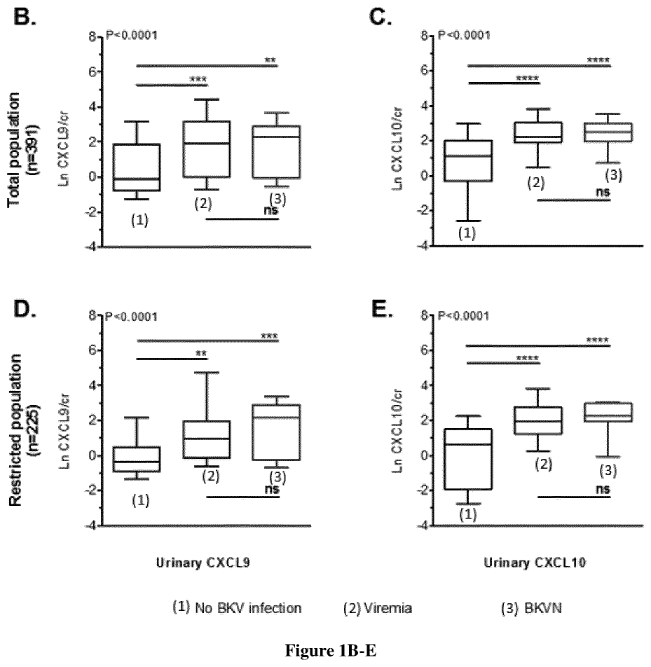

To further address whether different stages of BKV infection might impact urinary chemokine levels, we categorized the samples into three non-overlapping groups according to their BKV status (FIG. 1A).

In the whole population (N=391), the urinary levels of CXCL9 and CXCL10 (median [IQR], FIGS. 1B and 1C) remained low in the absence of BKV reactivation (LnCXCL9/cr=−0.1[−0.8-1.9], LnCXCL10/cr=1.13[−0.3-2.0]), while they were significantly increased in cases of isolated BKV viremia (LnCXCL9/cr=1.9[0.0-3.2], LnCXCL10/cr=2.21[1.9-3.0], P<0.001) and BKVN (LnCXCL9/cr=2.29[−0.1-2.9], LnCXCL10/cr=2.51[2.0-3.0], P<0.001). Interestingly, there were no significant differences in urinary chemokine levels between the BKV viremia-only group and the BKVN group (P>0.99, FIGS. 1B and 1C).

As BKV reactivation may occur concomitantly with UTI or AR, a sensitivity analysis was performed by excluding samples with significant leukocyturia (isolated or with UTI) and those with AR. In this restricted population (N=225), the results remain unchanged (FIGS. 1D and 1E).

Urinary Levels of CXCL9 and CXCL10 are Increased in UTI Cases but not in Isolated Leukocyturia Cases

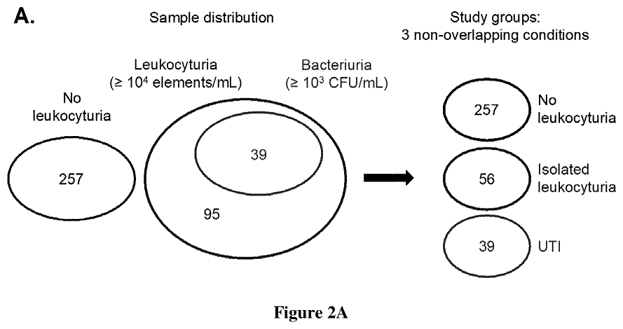

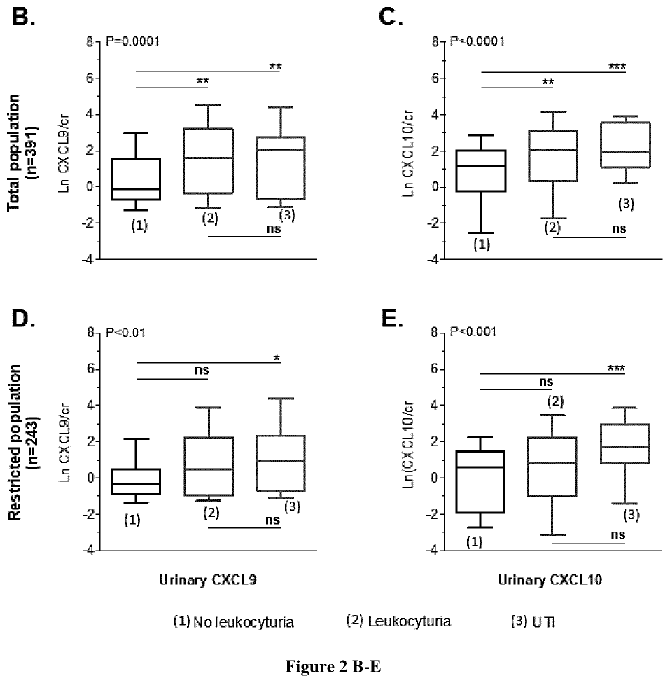

Next, we investigated the association between UTI and urinary chemokine levels. In the whole population, urinalysis identified 56 samples with isolated leukocyturia and 39 with UTI (FIG. 2A). Compared to the group of 257 samples with no leukocyturia, the groups with isolated leukocyturia or UTI exhibited significant increases in CXCL9 (median [IQR], LnCXCL9/cr: 1.61[−0.4-3.2] and 2.09[−0.6-2.8] vs −0.10[−0.7-1.5], P<0.01 for both, FIG. 2B) and CXCL10 (LnCXCL10/cr: 2.09[0.4-3.1] and 1.98[1.1-3.6] vs 1.17[−0.2-2.1], P=0.001 and P<0.001, respectively, FIG. 2C).

As previously described, a sensitivity analysis was performed by excluding cases of AR and cases of BKV viremia (with or without BKVN). In this restricted population (N=243, FIGS. 2D and 2E), compared to the samples with no leukocyturia, the samples with UTI (N=27) had significantly increased CXCL9 (P<0.01) and CXCL10 (P<0.001) urinary levels, contrary to samples with isolated leukocyturia (P=0.14 and P=0.80, respectively).

Construction of an optimized model for noninvasive diagnosis of acute rejection

Then, uCXCL9 and uCXCL10 were compared between samples with or without AR. In the total population (data not shown), chemokines were indeed significantly higher in AR than non-AR cases (LnCXCL9/cr: 1.92[−0.3-3.2] vs −0.10[−0.8-1.6], LnCXCL10/cr: 2[1.2-2.9] vs 1.12[−0.5-2.1], P<0.0001). In a sensitivity analysis after exclusion of BKV viremia (with or without BKVN) and leukocyturia (with or without UTI), CXCL9 and CXCL10 remained significantly increased in AR cases (data not shown).

Next, we performed a logistic regression analysis to build several models for noninvasive diagnosis of AR. Seventeen candidate variables were considered in the univariate analysis, and all variables with a P-value ≤0.25 (N=14) were entered into multivariable regression (Table 4). Two chemokines are highly correlated (Spearman r=0.60, 95% CI: 0.53-0.66, P<0.0001) raising the methodological issue of multicollinearity (data not shown). However, with a VIF well below the most conservative upper limit of 2.5, collinearity can be regarded as low, thus allowing us to keep both chemokines for further analysis (data not shown).

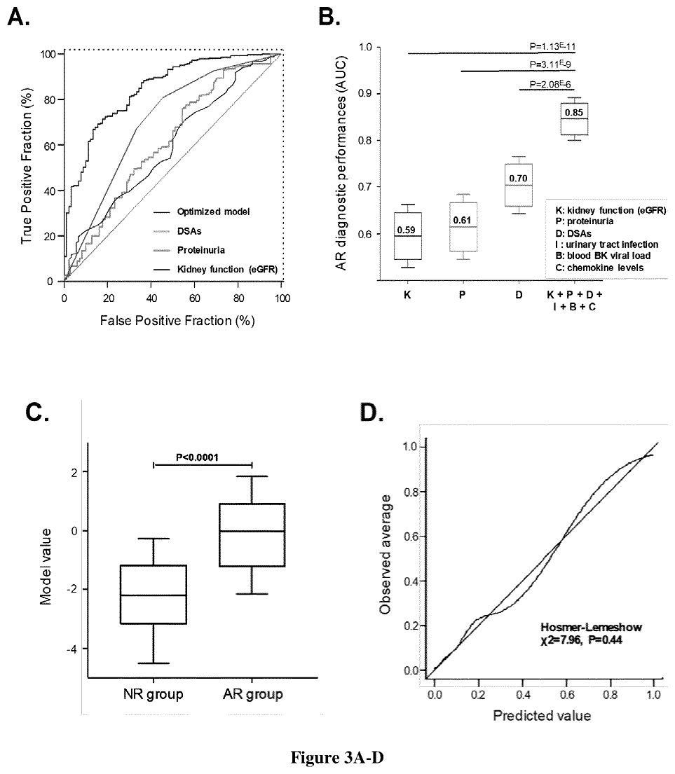

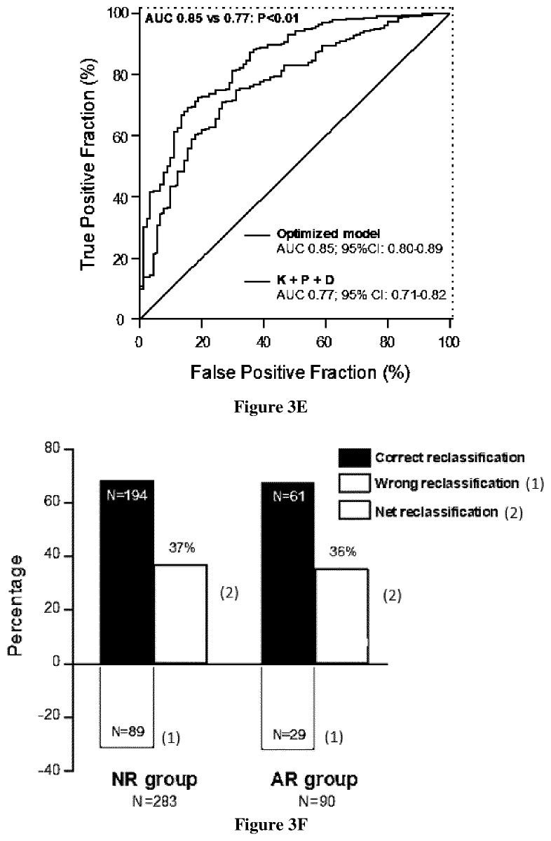

FIGS. 3A and 3B illustrate the poor diagnostic performance of the three parameters used in clinical practice to assess the risk of AR (i.e., eGFR, proteinuria and DSAs) eventually leading to graft biopsy. In comparison, combining eight variables among four categories (clinical variables [recipient age and sex], biological variables [eGFR and DSA score], confounding factors [BKV viremia and UTI]) and urinary chemokines (CXCL9/cr and CXCL10/cr) into an optimized mathematical model dramatically improved the assessment of AR risk (P<0.0001). Interestingly, though usually regarded as a relevant biological item in the context of AR, once adjusted for urinary chemokine levels in the multivariable analysis, proteinuria was not found to be independently significant (P=0.126).

The results of the bootstrapped logistic regression analysis of the final model are given in Table 3. The final multiparametric model strongly discriminated AR from non-AR samples (−0.02 vs −2.21, P<0.0001, FIG. 3C) with an AUC of 0.85 (95% CI: 0.80-0.89, P=2.43E-23), and the calibration curve showed good concordance between observed and predicted AR, as confirmed by the Hosmer-Lemeshow goodness-of-fit test (χ2=7.96, P=0.44, FIG. 3D).

Ultimately, eGFR, proteinuria and DSAs were entered into a statistical model to compute a decision-making model based on these 3 usual parameters. This clinical model, though far more advanced than current clinical practice, only slightly improved diagnostic accuracy (AUC: 0.77, 95% CI: 0.71-0.82, P<0.001, FIG. 3E). We assessed the value of the optimized model to reclassify the risk of AR, compared to this clinical model (FIG. 3F). In the non-AR group, the optimized model correctly reclassified 69% (N=194/283) of patients to a lower risk. In the AR group, appropriate reclassification to a higher risk was found in 68% (61/90) of AR patients. Overall, the optimized model improved classification both by correct net downward reclassification in patients without AR events (37%) and correct net upward risk reclassification in patients with AR events (36%).

Validation of the Optimized Model for Noninvasive Diagnosis of Acute Rejection

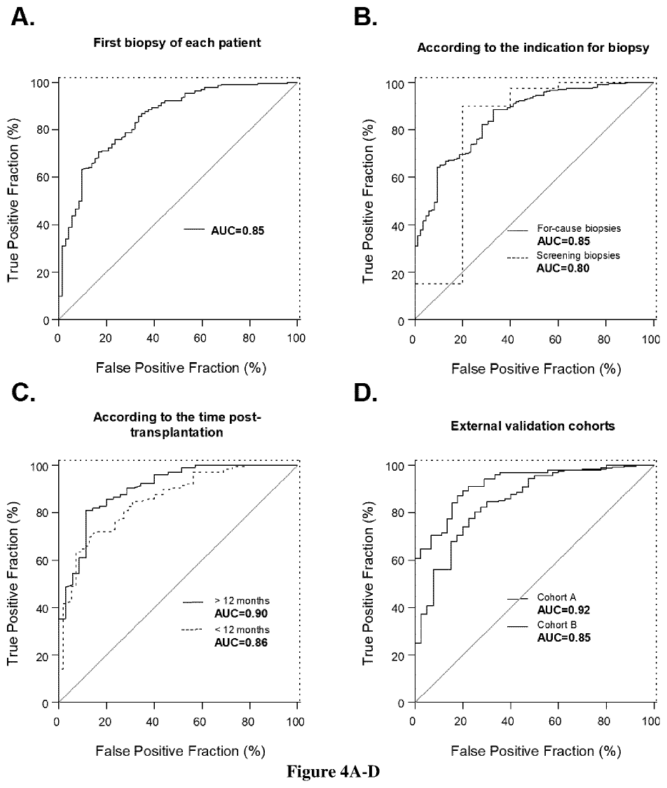

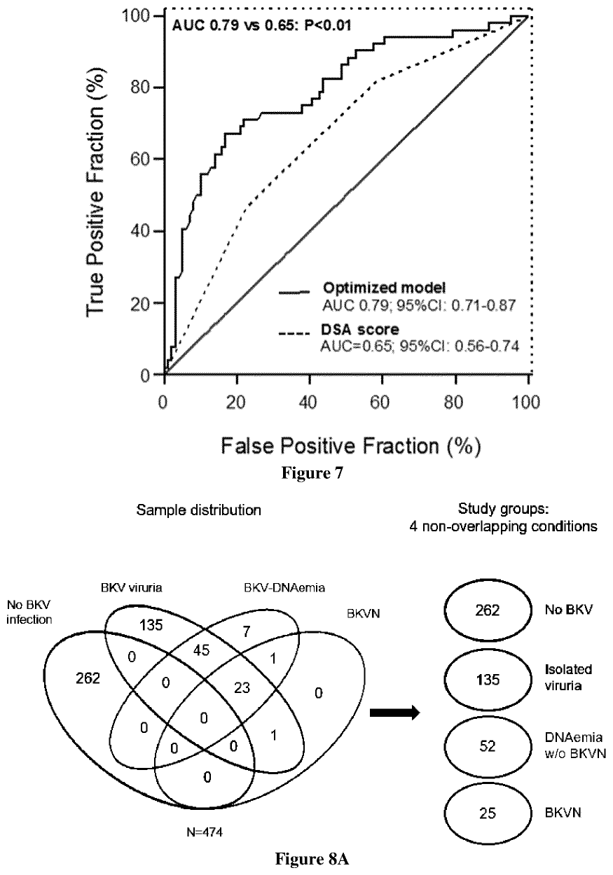

Internal validation of the model included several sensitivity analyses (FIG. 4A,B,C). The optimized model remained highly accurate when only the first biopsy of each patient was retained (AUC: 0.85, 95% CI: 0.80-0.90; P=1.4E-19), in cases of stable graft function (AUC: 0.81, 95% CI: 0.48-1; P=1.3E-02), at the time of graft dysfunction (AUC: 0.85, 95% CI: 0.81-0.90; P=1.2E-22), and within the first year (AUC: 0.86, 95% CI: 0.80-0.91; P=9E-16) or later (AUC: 0.90; 95% CI: 0.84-0.96; P=5.9E-13) after transplantation. Moreover, a subset analysis was performed in KTR with pre-existing or de novo DSA, to assess the benefit of the model to detect antibody-mediated rejection (ABMR). In comparison to the DSA score alone, the optimized 8-variable model significantly improved ABMR diagnosis accuracy (0.79 vs 0.65, P<0.01, FIG. 7).

In addition, robustness of the optimized model was assessed in two external validation cohorts. First of all, Cohort A included 147 urine specimens collected at time of mainly indication biopsies (75.5%) in 109 single-center KTRs. In addition, Cohort B included 295 urine specimens collected at time of mainly screening biopsies (73.2%) in 282 KTRs from four European centers. The same 8 parameters diagnosed AR with an AUC of 0.92 (95% CI: 0.88-0.97; P<0.0001) in cohort A, and 0.85 (95% CI: 0.78-0.91; P<0.0001) in cohort B (FIG. 4D).

Accuracy Metrics of the Optimized Model for Noninvasive Diagnosis of Acute Rejection

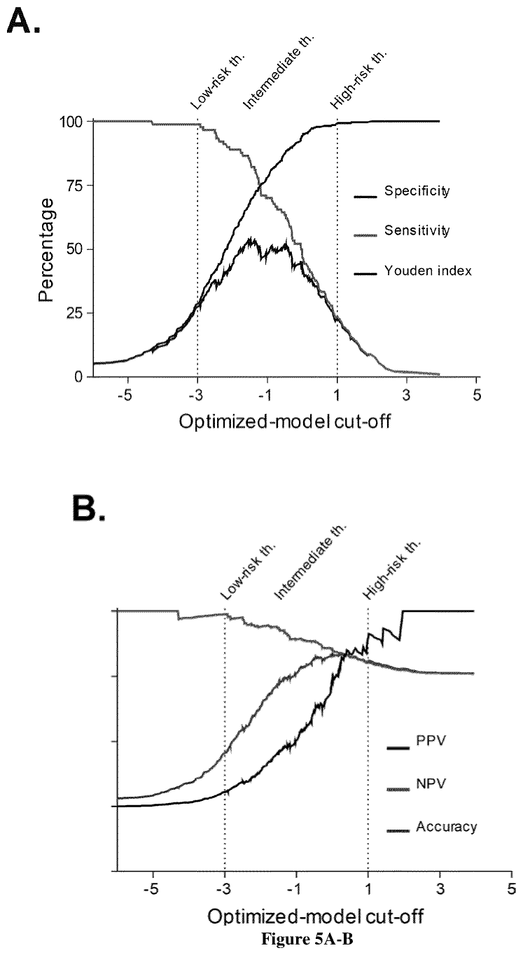

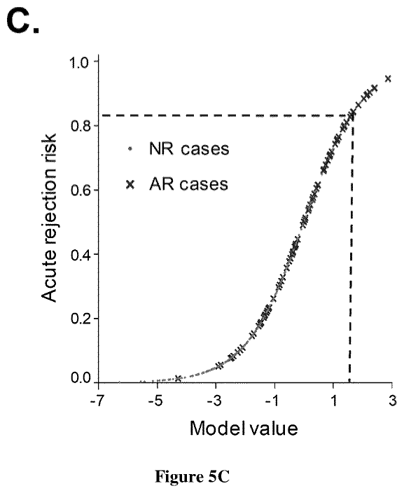

We next examined the cut-off value of the optimized multiparametric model. FIGS. 5A and 5B shows the accuracy metrics of the model. The optimal cut-off (−1.48) had a sensitivity of 84.4%, a specificity of 69.3%, PPV of 46.6%, and NPV of 93.3%. To adapt to various contexts of use, we defined two arbitrary thresholds: a “low risk” cut-off of −3, which optimized sensitivity and NPV by reducing the fraction of false negative results and a “high-risk” cut-off of +1, which maximized specificity and PPV by reducing the fraction of false positive results.

This optimized multiparametric non-invasive model provides a risk of AR at an individual level. Probability (p) of acute rejection can be computed from the following equation (with each β coefficient of each variable (x) as defined in Table 5):

$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

As an example, a 43-year-old woman with a eGFR of 20 mL/min, the presence of DSAs with an MFI of 1811, no UTI, no detectable BK viremia, uCXCL9/cr level of 3.04 ng/mmol, and uCXCL10/cr level of 20.65 ng/mmol had a value of 1.45, which corresponds to a predicted risk of AR of 81% (FIG. 5C).

Finally, practical or financial considerations could stimulate the use of a slightly degraded model with only one of the two chemokines. As shown above, collinearity is low (data not shown) and both chemokines were selected in the multivariable logistic regression (Table 3) indicating that both provide specific information. The performances of CXCL9 and CXCL10 alone, in combination, or included in multiparametric models for the diagnosis of AR, T-cell mediated and antibody-mediated rejections (data not shown).

Clinical Utility of the Urinary Chemokine Model in Optimizing the Cost/Benefit Ratio of Biopsies

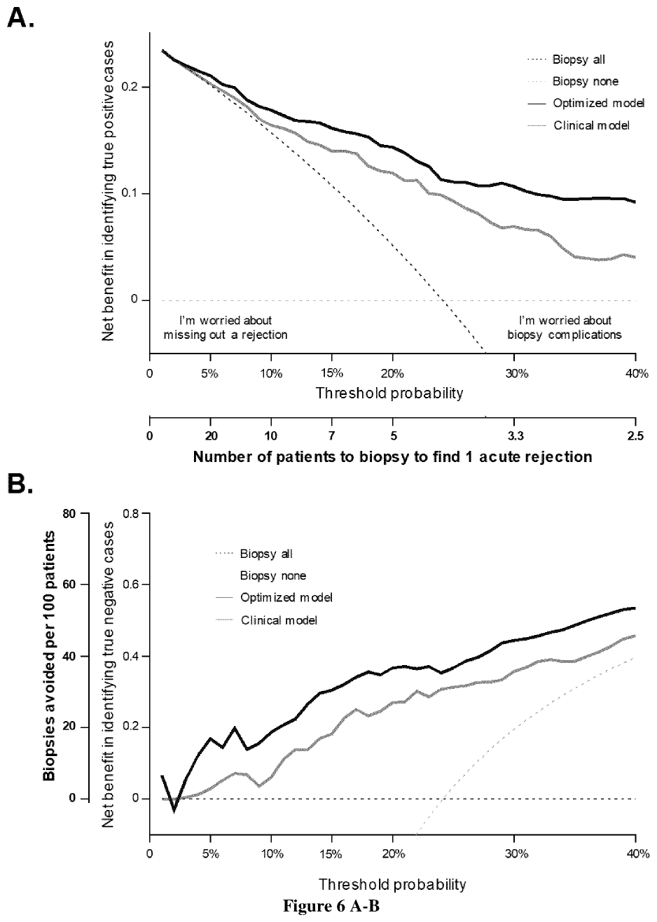

Traditional statistical measures for the evaluation of prediction models (accuracy metrics, discrimination, calibration) do not provide an answer as to whether the model should be used in clinical practice. To put benefits (identifying AR) and harms (biopsy complications and/or costs) on the same scale¹⁴, we performed a decision curve analysis, which calculates a clinical “net benefit” for one or more prediction models in comparison to default strategies of performing a biopsy to all or no patients. FIG. 6A shows the net benefit (identifying true positive cases) of the optimized model compared to the clinical model (eGFR, proteinuria, DSAs) according to the threshold probability. The threshold probability varies according to clinicians and patients' preferences and can be better understood if considered as “biopsies performed to find one rejection” (see secondary x-axis). The blue line, corresponding to the optimized model, has the highest benefit across a wide range of reasonable threshold probabilities. FIG. 6B shows the “net benefit” expressed as biopsies avoided (secondary y-axis), corresponding to true negative cases. Again, the optimized model has a high benefit across a wide range of risks. As an example, at the threshold risk of 10% (“I would not want to do more than 10 biopsies to find one acute rejection”), 13 biopsies would be avoided per 100 patients if using the optimized model compared to the clinical model.

The urinary chemokine model could be used across an individual patient scenario. Routine graft status work-up (serum creatinine, proteinuria, DSAs, sonography, calcineurin inhibitors trough level) defines the context of use (data no shown). In unstable patients, the net benefit of the model was assessed compared to a default strategy of performing a biopsy on all patients (FIG. 7, upper panel). At the threshold risk of 10%, the number of unnecessary biopsies avoided was 17 per 100 unstable patients. In stable patients, in a transplant center where no screening biopsy is performed, the model would help to identify 6 out 100 apparently stable patients who would benefit from a biopsy to diagnose a subclinical rejection. In a transplant center with screening biopsy as part of routine work-up, the model could help to avoid 58 unnecessary biopsies out of 100 stable patients (FIG. 7, lower panel).

In conclusion, UTI and BKV viremia (with or without BKVN) are associated with increased urinary concentrations of CXCL9 and CXCL10. Rather than excluding these confounding factors, we incorporated them in an optimized multiparametric diagnostic model for AR of kidney allografts. This model, which includes easily available clinical and laboratory data and results from simple ELISA tests for CXCL9 and CXCL10, achieves unprecedented accuracy for a noninvasive diagnostic tool.

### Example 2

We aimed to investigate urinary C—X—C motif chemokine 10 (uCXCL10) as a diagnostic biomarker in the course of BKV infection by determining uCXCL10 levels across different stages of BKV replication, evaluating its potential as a prognostic biomarker in comparison to conventional biological and histological markers, and describing the longitudinal course of uCXCL10 in BKV infection.

Material & Methods

Population

Cross-sectional study (data not shown). We retrospectively considered all adult KTRs followed at Necker Hospital (Paris, France) between February 2011 and February 2016 and selected N=474 samples collected from N=391 patients with concomitant (i) blood BKV viral load measurements obtained within ten days before or after allograft biopsy, (ii) informative allograft biopsy, and (iii) available urine samples for research use. Among patients with BKV-DNAemia (N=76), we retained only the first sample from each patient for the nested case-control study (N=63).

Longitudinal study (data not shown). Serial measurement of urinary chemokines became part of routine follow-up during the first year post-transplant at Necker Hospital, starting in April 2017. Urine samples for uCXCL10 quantification were collected at biopsy and at each outpatient clinic visit. We retrospectively considered all consecutive adult patients with at least one positive BKV-DNAemia test from April 2017 on, with a minimum of six months of follow-up (N=60). Of those 60 individual patients, 46 patients had a urinary chemokine assessment within 7 days before or after their 1st positive BKV-DNAemia. The study was approved by the Ethics Committee of Ile-de-France XI (#13016), and all participating patients provided written informed consent.

Urine Sample Collection

Urine specimens were collected (immediately before the allograft biopsy if any) and centrifuged at 1000×g for 10 minutes at 4° C. within 4 hours of collection. The supernatant was collected after centrifugation and stored with (cross-sectional study) or without (longitudinal study) protease inhibitors (cOmplete™, Roche Diagnostics, Meylan, France) at −80° C. Urine cell pellets were resuspended in 1 mL of phosphate-buffered saline and then centrifuged for 5 minutes at 12000× g at room temperature. The supernatant was eliminated, and urine cell pellets were resuspended in RLT Buffer (RNeasy® Mini Kit, Qiagen, Courtaboeuf, France) and stored at −80° C.

Urine Protein Analyses

Frozen aliquots of urine supernatants were thawed at room temperature immediately before ELISA. Samples were used without dilution and tested in replicate analysis. CXCL10 (Human CXCL10/IP10 Quantikine ELISA kit, Bio-Techne, Minneapolis, USA) was quantified according to the manufacturer's instructions. Optical densities were derived from a 4-parameter logistic regression of the standard curve. The results were normalized to the urinary creatinine level through determination of the uCXCL10/cr ratio (nanograms of protein per millimole of urinary creatinine).

For the cross-sectional study, ELISA was performed manually, and optical densities were measured using a Multiskan FC plate reader (Thermo Fisher, Illkirch, France). Urine samples with a chemokine concentration below the mean minimum detectable level in the ELISA assay (0.8 pg/mL) were included in the analysis as one-half the detection limit. Measurement of creatinine in urine was performed in the same samples using the Creatinine Parameter Assay Kit (Bio-Techne).

For the longitudinal study, ELISA was performed using an EVOLIS™ Twin Plus System (Clinical Diagnostics, Bio-Rad, Marnes-la-Coquette, France). One-half of the detection limit was 1.95 pg/mL. Measurement of creatinine in urine was performed in the same sample using an Architect c8000 and C16000 (Abbott Diagnostic, Rungis, France).

BK Virus Analyses

To assess the urine BKV viral load, we used gene-specific oligonucleotide primers and probes (Thermo Fisher) to measure messenger ribonucleic acid (mRNA) encoding BKV VP1 capsid protein as previously reported (35). We used previously published predeveloped and custom-synthetized primers and probes. Total RNA was isolated from urine cell pellets using an RNeasy Mini Kit (Qiagen). RNA concentration was determined using a NanoDrop-2000 spectrophotometer (Thermo Fisher, Montigny le Bretonneux, France). For comparison purposes, RNA samples were concentrated by evaporation for 30 minutes at 60° C. using a SpeedVac™ (Thermo Fisher) and then resuspended at the same concentration of 10 ng/μL in reverse transcription (RT) mix (Taqman Reverse Transcription Reagents, Thermo Fisher). RT was performed on a Veriti® Thermal Cycler (Thermo Fisher) with the following program: 10 minutes at 25° C., 30 minutes at 48° C., and then 5 minutes at 95° C. Then, 1.5 μL of cDNA (after 1/1000 dilution of RT product for BKV expression) was used for qPCR assay performed in replicate analysis on a Viia™ 7 Real-Time PCR System (Thermo Fisher) using a Fast protocol: 95° C. for 20 seconds, followed by 40 cycles of amplification (95° C. for 3 seconds, 60° C. for 30 seconds). Absolute quantification of gene expression was performed using the murine gene BAK as a standard, with a known number of copies of RNA per μg, with the final result expressed as the number of copies per nanogram of total RNA. A second assay was performed when inconsistent results were obtained (N=7 cases negative for BKV viruria and positive for BKV-DNAemia).

BKV-DNAemia in whole blood samples was monitored in our hospital laboratory by real-time qPCR (BK Virus R-gene, BioMérieux®, Marcy l'Etoile, France) with a positive threshold value of 2.4 Log10 copies per mL (500 copies/mL), the lower limit of detection for the assay.

The different stages of BKV reactivation were defined as follows: the no BKV infection group, viruria group (viruria detected with no BKV-DNAemia or BKVN), DNAemia group (positive for BKV-DNAemia, regardless of BKV viruria, in the absence of biopsy-proven BKVN) and BKVN group (positive SV40 staining and/or viral inclusion on biopsy specimen).

Histology

Biopsy specimens were fixed in formalin, acetic acid and alcohol and embedded in paraffin. Tissue sections were stained with hematoxylin and eosin, Masson's trichrome, periodic acid-Schiff reagent, and Jones stain for light microscopy evaluation. C4d immunohistochemical staining was systematically performed (with rabbit anti-human monoclonal anti-C4d; 1/200 dilution; Clinisciences, Nanterre, France). Clinically indicated or for-cause biopsies were classified using the 2015 update of the Banff 1997 classification (36).

All biopsies performed in patients with concomitant BKV-DNAemia were reviewed by two investigators (MR, AV), and SV40 immunohistochemical staining was systematically performed (anti-SV40 T Antigen Mouse mAb (PAb416), Calbiochem®, USA).

Donor-Specific Antibodies

All circulating donor-specific anti-human leukocyte antigen antibodies (DSAs) were determined with single-antigen flow bead assays (One Lambda, Canoga Park, USA) on a Luminex platform in a single laboratory (Saint-Louis Hospital, Paris). Beads showing a normalized mean fluorescence intensity greater than 500 were considered positive.

Urinary Tract Infection

Cytobacterial examination of urine was systematically performed at the time of urine collection. Urinary tract infection (UTI) was defined by bacteriuria ≥103 colony-forming units (CFU) and leukocyturia ≥104 white blood cells per mL. Both symptomatic and asymptomatic UTIs were included.

Statistical Analyses

The results are presented as the mean±standard deviation (SD) for continuous variables, except for time from transplant to biopsy, viral loads and CXCL10 levels, which are presented as the median and interquartile range [IQR]. The frequencies of categorical variables are presented as numbers and percentages. The distribution of uCXCL10/cr exhibited considerable positive skew, which was substantially reduced by use of natural logarithm (1n) transformation. We compared groups using the Mann-Whitney test or Kruskal-Wallis test followed by Dunn's post-test, when appropriate. We compared proportions using Fisher's exact test or a Chi-2 test with Yate's continuity correction when appropriate. We used a parametric Pearson correlation test on log-transformed variables when the sample size was >30. P-values ≤0.05 were regarded as statistically significant.

To identify parameters independently associated with allograft prognosis after BKV-DNAemia, death-censored Cox regression analysis (Table 7) was performed. We tested for biologically and histologically relevant variables for explaining a given decrease in graft function. Natural logarithm transformation was used to reduce right skewness. A multivariate model was built, including all variables with P<0.2 in the univariate analysis. With a 50% eGFR decrease event occurring in 23 patients, a minimum of “10 events per variable” was respected, with no more than three variables entered in the final multivariate regression. The Kaplan-Meier method was used for the survival analyses.

A random forest classification analysis was also used to address the importance of variables in explaining allograft prognosis after BKV-DNAemia. Out-of-bag error (the error rate from samples not used in the construction of a given tree) was minimized by tuning the number of trees (ntrees=1000) and the number of variables randomly chosen at each node (mtry=4). The results are given as a variable importance measure according to the mean Gini decrease.

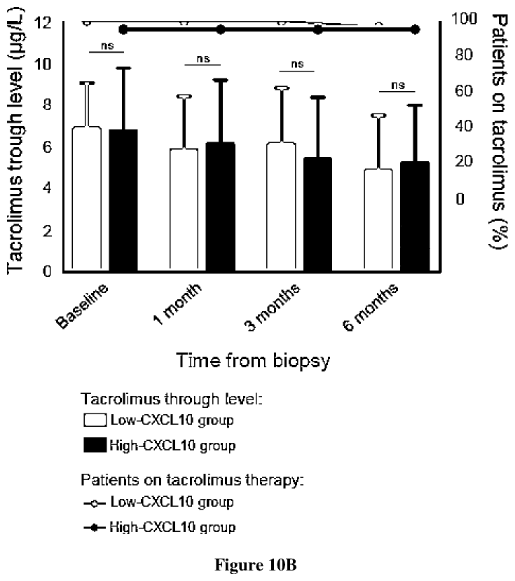

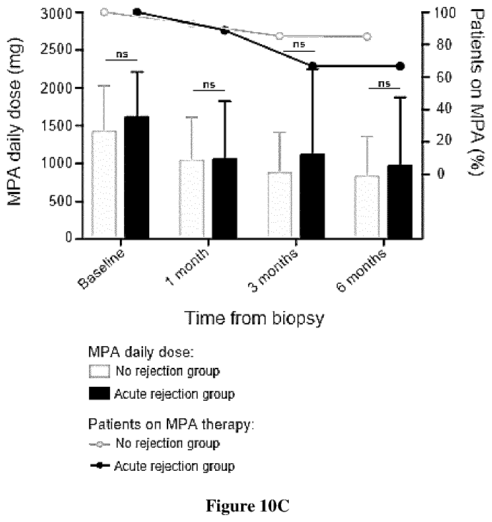

Finally, to compute the trajectory analyses (FIG. 10A, 10B), the k-nearest neighbors method was used for local regression of the longitudinal data (blood BKV viral load and uCXCL10/cr), followed by modeling of the regression for the entire sample period. The urinary CXCL10/cr area under the curve (AUC) was calculated for each patient from first BKV-DNAemia to BKV negativity, censored by the rejection date, if any. To reflect the intensity of the immune response and not the time to BKV clearance, the AUC was normalized by the number of days of BKV-DNAemia and thereupon expressed as time-adjusted uCXCL10/cr AUC (ng/mmol/d). In this analysis, DNAemia lower than the limit of quantification (LOQ) was included as one-half of the LOQ.

Outcomes were determined as of Nov. 29, 2019, for the cross-sectional cohort and as of Apr. 20, 2020, for the longitudinal cohort.

Analyses were performed with R software (R Development Core Team, R version 3.6.3 and R studio version 1.2.5033) and GraphPad PRISM® Software GraphPad Software, San Diego, USA, version 7.0a).

Results

Cross-Sectional Cohort

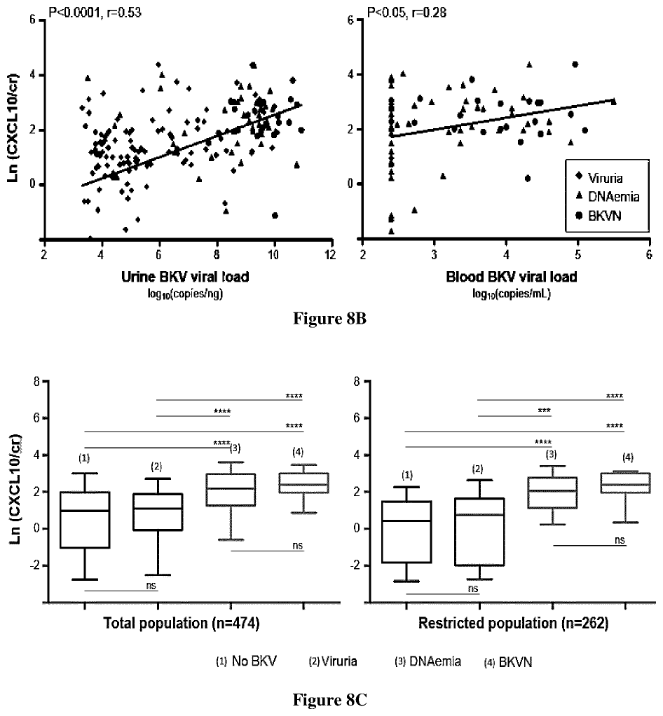

In this study, 474 sets of three samples (i.e., allograft biopsy/urine/blood) collected from 391 individual patients were identified (data not shown). Biopsies were performed at a median time of 11 [IQR: 34] months post-transplantation and were mainly clinically indicated (87.8%, Table 6). At the time of biopsy, the mean serum creatinine was 185±99 μmon. BKV-DNAemia was detectable in 16% of cases, with a median viral load of 3.32 [IQR: 3.1] Log10 copies/mL, and 5.3% met the criteria for BKVN (N=25). BKV viruria was detectable in 43% of cases, with a median viral load of 7.2×105 [IQR: 6.8×108] Log10 copies/ng. As expected, BKV viruria was found in most BKV-DNAemia (86.5%) and BKVN samples (96%). To further address whether different stages of BKV infection might impact uCXCL10 levels, we categorized samples into 4 non-overlapping groups (FIG. 8A) according to their BKV status: the no BKV infection group (N=262 samples), viruria group (N=135), DNAemia group (N=52) and BKVN group (N=25).

Urine Levels of CXCL10 are Significantly Correlated with Urine and Blood BKV Viral Load

We next investigated urine BKV viral load and its correlation with uCXCL10/cr levels. As shown in FIG. 8B, the median level of BKV viruria gradually increased in the viruria (7.3×104 Log10 copies/ng), DNAemia (6.8×108 Log10 copies/ng) and BKVN (3.6×109 Log10 copies/ng) groups and was strongly correlated with uCXCL10/cr levels (P<0.0001, Pearson r=0.53). Similarly, the median BKV-DNAemia was higher in the BKVN group than in the BKV-DNAemia group (3.96 vs 2.68 Log10 copies/mL, P<0.001) and also correlated with uCXCL10/cr to a lesser extent (P<0.05, Pearson r=0.28, FIG. 8B, right Panel). As previously reported (37), BKV loads in the urine and blood were correlated (P<0.01, data not shown).

Urine Levels of CXCL10 are Similarly Increased in BKV-DNAemia and BKVN but not in Isolated BKV Viruria

In the whole population (N=474), uCXCL10/cr (FIG. 8C, left Panel) remained low in the viruric group as in the absence of BKV reactivation (1.12 vs 0.98, P>0.99). As expected, uCXCL10/cr was significantly increased in patients with BKV-DNAemia with or without biopsy-proven BKVN (both P<0.0001). Interestingly, there was no significant difference in chemokine levels between the BKV-DNAemia group and the BKVN group (P>0.99). In a sensitivity analysis, we excluded samples with suspected urinary tract infection (or no cytobacterial examination of the urine, N=98) and those with concurrent acute rejection (or inadequate biopsy, N=135), all established confounding factors for increased uCXCL10/cr (34). In this restricted population (N=262) where the uCXCL10/cr is hypothesized to be only driven by BKV infection, the results remained unchanged (FIG. 8C, right panel).

Finally, we identified 25 patients from the viruric group with a unexpected high uCXCL10 level. Of those, 80% actually have concurrent acute rejection or UTI, and only few remained in the restricted population (data not shown). For the 5 remaining cases, we cannot exclude that other confounders were missed or that the intensity of BKV replication within the urinary tract led to a significant inflammatory response.

Urinary CXCL10 Levels in Patients with BKV-DNAemia is a Prognostic Marker of Allograft Function

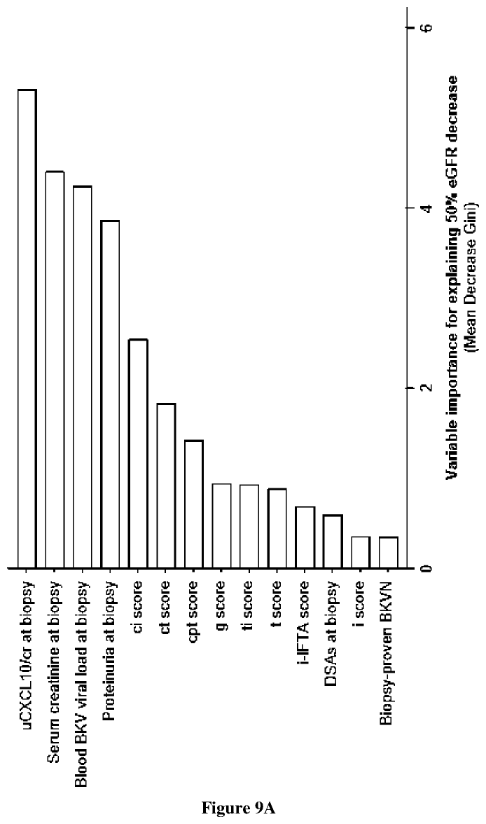

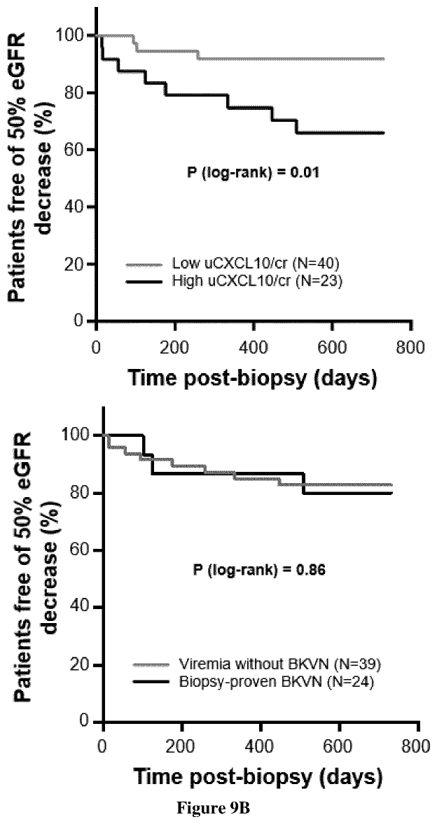

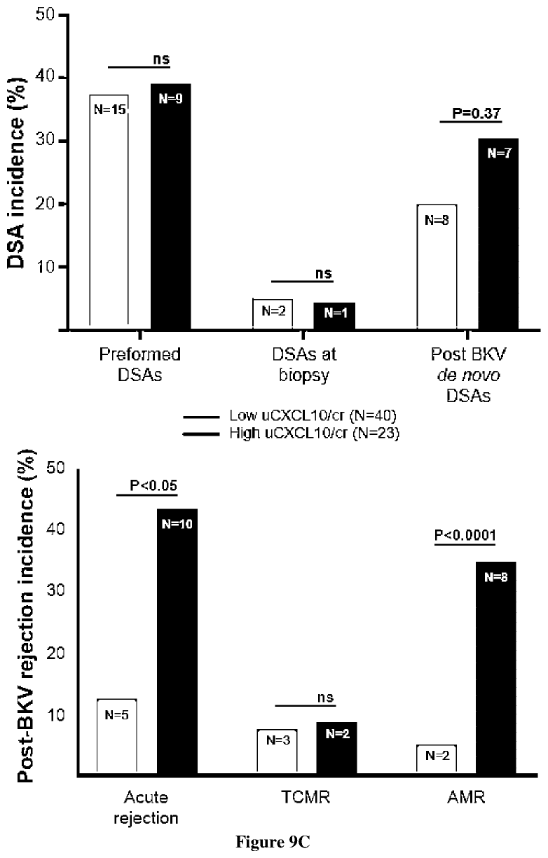

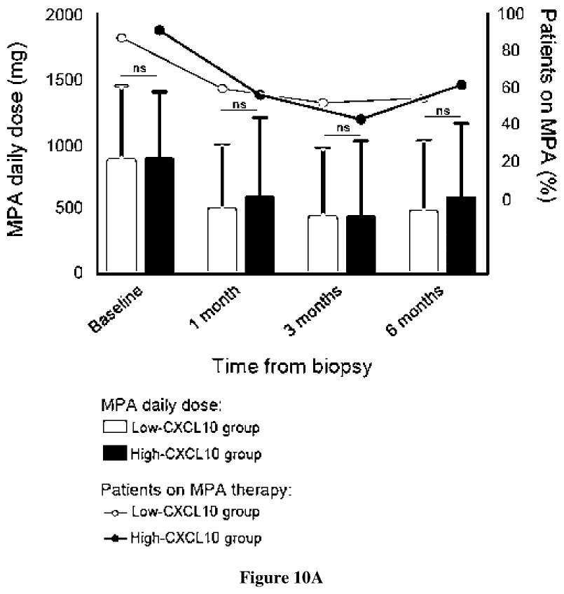

In the nested case-control study, we focused on renal allograft outcomes among KTRs with BKV-DNAemia, with or without BKVN. We retained only the first sample from each patient, leading to N=63 unique patients with the set of three samples (i.e., biopsy/urine/blood). The median time from transplantation to biopsy was 10 [IQR: 19] months, and the median follow-up time was 55 [IQR: 38] months. Nine patients (17%) had a concurrent diagnosis of acute rejection. The determinants, at the time of biopsy, of subsequent allograft function worsening, as assessed by a 50% eGFR decline, were studied by random forest analyses and a Cox proportional hazard model. The random forest analysis (FIG. 9A) reveals the relative variable importance for explaining the renal outcome (mean decrease in Gini score), allowing visual comparison the respective weight of each explicative variable. As expected, blood viral load, kidney function, and proteinuria were relevant in explaining graft decline. However, uCXCL10/cr clearly outperformed these biological variables as well as the histological diagnosis of biopsy-proven BKVN. Consistently, the multivariate Cox model confirmed uCXCL10/cr as the unique determinant of subsequent graft function decline (HR=1.52, 95% CI [1.00-2.30], P<0.05), independent of allograft function (P=0.46) and blood viral load (P=0.50) at the time of biopsy or the presence of biopsy-proven BKVN (P=0.84) (Table 7). A threshold of 12.86 ng/mmol discriminated patients with a low risk of postbiopsy graft function decline from the high-risk group (FIG. 9B, log-rank P-value=0.01). In contrast, the same survival analysis comparing BKV-DNAemia with and without BKVN showed no difference (log-rank P-value=0.86).

Allograft Rejection Drives the Evolution of Renal Function after BKV-DNAemia

As our multivariate analysis identified uCXCL10/cr at the time of biopsy as an independent predictor of postbiopsy graft dysfunction, we aimed to identify the underlying determinants that link uCXCL10/cr at the time of biopsy to postbiopsy graft dysfunction. At the time of biopsy, the patients with low and high uCXCL10/cr levels were similar with regard to eGFR (P=0.83), BK blood viral load (P=0.59), peak viral load (P=0.16), primary histological diagnosis of acute rejection (P=0.36) and BKVN (P=0.55).

BKV-DNAemia led to tapering of the immunosuppressive regimen, with no significant difference between the two groups with regard to mycophenolic acid daily dose or tacrolimus trough levels at baseline, 1-3 and 6 months after biopsy (FIGS. 10A and 10B).

De novo DSAs occurred in 30.4% of patients in the high-CXCL10 group compared to 20% of those in the low-CXCL10 group, but this difference did not reach significance (P=0.37, FIG. 9C). However, within a median time of 6 months postbiopsy, acute rejection occurred significantly more often in the high-CXCL10 group than in the low-CXCL10 group (P<0.05), consisting of mainly AMR (34.8% vs 5%, P<0.0001, FIG. 9C). Of note, baseline ABMR frequency was similar between both groups (P=0.25). Most importantly, 80% of rejection cases occur de novo with only 3 recurrent/persistent rejections (data not shown). Altogether, this information suggests that clinical prognosis relies on uCXCL10 and/or subsequent ABMR rather than baseline concurrent rejection.

Longitudinal Cohort: Validation of uCXCL10 Cut-Off as a Prognostic and Predictive Biomarker

Starting in April 2017, serial measurement of uCXCL10 became part of routine follow-up during the first year post-transplantation at Necker Hospital. We retrospectively considered all consecutive adult patients with at least one positive BKV-DNAemia test from April 2017 on, with a minimum of six months of follow-up. In this longitudinal cohort, 60 patients experienced BKV-DNAemia, within a median time of 5 [IQR: 7.3] months after transplantation.

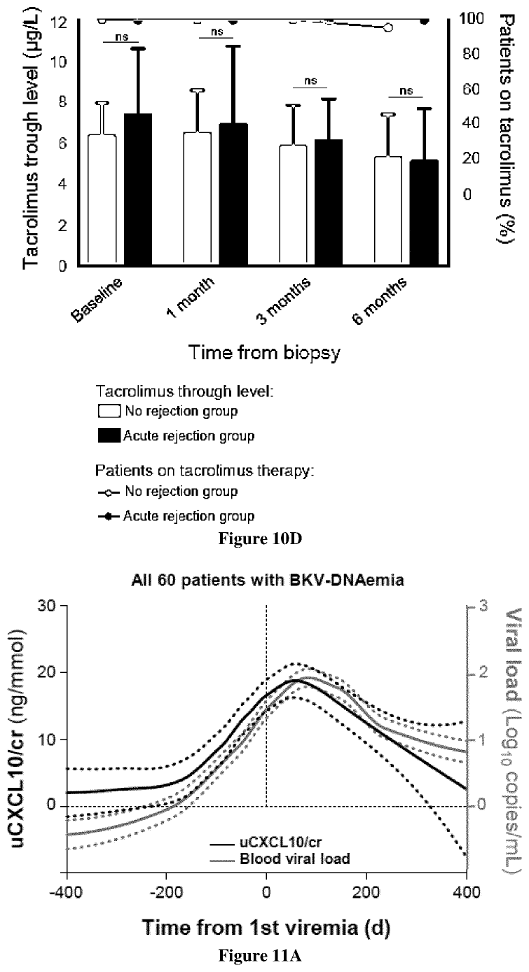

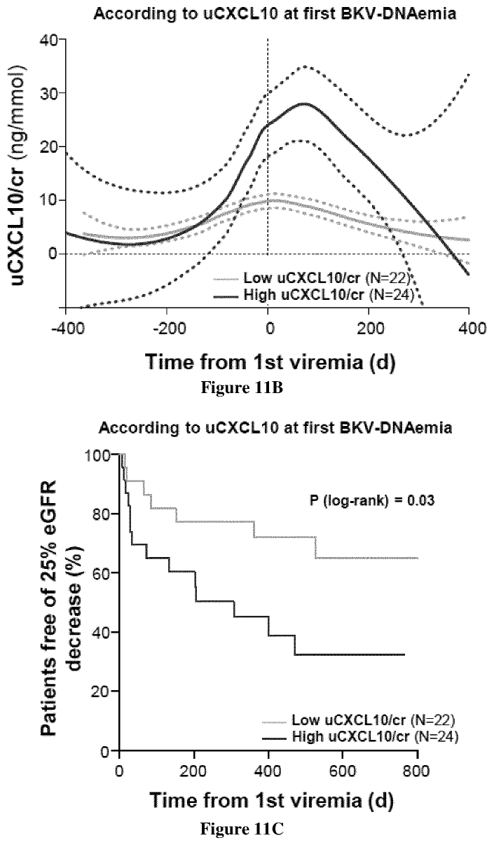

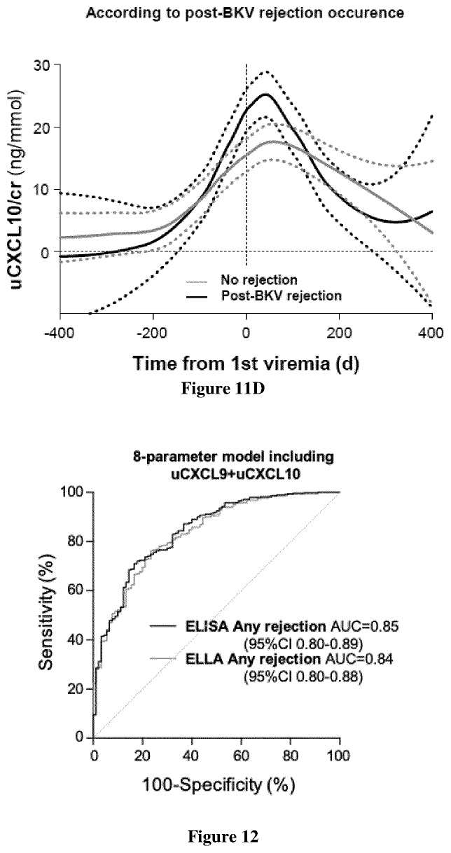

Using regression analyses, we computed the longitudinal trajectories of blood BKV viral load and uCXCL10/cr quantified in 1184 urine samples from these 60 patients. As depicted in FIG. 11A, the course of uCXCL10/cr parallels that of BKV-DNAemia throughout infection, with concomitant onset and resolution. Moreover, the longitudinal cohort was split in three groups, according to the latest guidelines (33). uCXCL10/cr gradually increased among transient, sustained low-level and sustained high-level DNAemia groups. However a larger sample size would be needed to reach significance in predicting the subsequent DNAemia profile, i.e. in identifying from the very first viremia, KTRs who could benefit from lowering of maintenance immunosuppression.

Next, we sought to validate the relevance of the uCXCL10/cr threshold (12.86 ng/mmol) established in the nest case-control cohort as a prognostic biomarker for allograft function. The longitudinal cohort was split into two groups according to uCXCL10/cr levels at the 1st BKV-DNAemia (≤ or >12.86 ng/mmol). The low- and high-CXCL10 groups had similar characteristics at first BKV-DNAemia, regarding median time post-transplantation (P=0.24), BKV viral load (P=0.25), and eGFR (P=0.18) (Table 8). In contrast, the uCXCL10/cr threshold at the first BKV-DNAemia identified two distinct populations regarding the outcome of the urinary inflammatory response (FIG. 11B). Patients with uCXCL10/cr ≤12.86 ng/mmol at first BKV-DNAemia had consistently low uCXCL10/cr levels throughout the follow-up period. In contrast, patients with uCXCL10/cr >12.86 ng/mmol at first BKV-DNAemia experienced a sharp peak in uCXCL10/cr (36.0 [31.2] vs 12.3 [13.4] ng/mmol, P<0.0001). To quantify the urinary inflammatory burden, we computed the area under the uCXCL10/cr curve (AUC) during BKV infection, normalized by the number of days. The time-adjusted uCXCL10 AUC was 19.3 [20.1] in the high-CXCL10 group compared to 7.15 [6.9] ng/mmol/d in the low-CXCL10 group (P<0.001). These two inflammatory patterns were not associated with BKV clearance time (132 [148] vs 131 [205] days, P=0.49) or peak BKV viral load (3.6 [1.3] vs 3.6 [1.2], P=0.89).

Besides, the same uCXCL10/cr threshold discriminated patients with a low risk of 25% eGFR decrease from high-risk patients (FIG. 11C, log-rank P-value=0.03). In a Cox proportional hazards regression model including uCXCL10/cr, eGFR and BKV viral load (all measured at the time of first BKV-DNAemia), uCXCL10 was significantly associated with 25% eGFR decrease (P-value<0.05, HR 1.733 [1.0076-2.9807]).

Ultimately, after resolution of BKV-DNAemia, acute rejection occurred in 9 patients. No difference was observed between the low-CXCL10 and high-CXCL10 groups (respectively 13.64% and 16.67%, P>0.99). However, the time-adjusted uCXCL10 AUC (censored by the rejection date) was higher in the group with subsequent rejection than in the immune-quiescent group (15.5 [11.7] vs 7.49 [6.4] ng/mmol/d, P=0.05, FIG. 11D), suggesting a stronger inflammatory response occurs during BKV infection prior to rejection, independent of immunosuppressive regimen weaning (FIGS. 10C and 10D).

In conclusion, in this study, we specifically addressed the performance of uCXCL10 in the course of BKV reactivation in KTRs. We show that uCXCL10 is not only increased at the time of BKV-DNAemia but also a robust prognostic marker for allograft function. We show that uCXCL10 outperforms many conventional biological and histological parameters, including blood viral load and biopsy-proven BKVN, in predicting the evolution of eGFR. Finally, uCXCL10 is a predictive biomarker, discriminating between different inflammatory responses to BKV infection, with the strongest inflammation eventually leading to eGFR decrease or acute rejection.

### Example 3

We evaluated the Ella® platform as a feasible technique for routine implementation of urine chemokine monitoring in kidney transplantation, and validated each workflow step from sample collection to results reporting. More precisely, we investigated preanalytical sample processing from collection to storage and Ella® analytical performance from urine sample, as to provide suggestions of standard operating procedures, an essential factor in ensuring excellent sample quality and reliable results. For clinical validation, we assessed whether the Ella® platform provided close and useful results in comparison to the reference ELISA technique. Finally, we reasoned that only a web application could advance the use of urine chemokine risk assessment for acute rejection to daily care, and build a web app calculator as a handy tool for clinicians' use as of now.

Material & Methods

Study Samples and Cohorts

Samples for Ella® technical validation (preanalytical and analytical performance studies) were all taken from the local Transplantation Biobank (Necker Hospital, Paris, France). Urine samples were routinely collected from kidney transplant recipients as part of transplant care, with exception for the “storage study” where freshly emitted urine samples (N=12) were prospectively collected. Besides, urine samples (N=10) were collected from kidney living-donors at the time of their pre-donation evaluation.

Samples for the clinical validation studies belong to previously published cohorts: Cohort A comprised 275 samples and Cohort B comprised 372 samples. A full description of their clinical and biological characteristics is available in Rabant et al. J Am Soc Nephrol 2015 and Tinel et al. Am J Transplant 2020. The study was approved by the Ethics Committee of Ile-de-France XI (#13016), and all participating patients provided written informed consent.

ELISA Methods

Extensive protocol for urinary chemokines quantification by enzyme-linked immunosorbent assay (ELISA) has been described somewhere else (Tinel C, Am J Transplant 2020). Briefly, uCXCL9 was measured using Human CXCL9/MIG DuoSet ELISA kit (Bio-Techne, Minneapolis, USA), with a protocol optimized for quantification from a urine sample. Human CXCL10/IP10 Quantikine ELISA Kit (Bio-Techne,) was used according to the manufacturer's instructions. Optical densities were measured using a Multiskan FC plate reader (Thermo Fisher, Illkirch, France). All measurements were performed in duplicate.

Ella® Immunoassay Methods

Urine CXCL9 and CXL10 levels were measured using the Ella® microfluidic Single Plex cartridges (ProteinSimple™, San Jose, Calif.), following the manufacturers' instructions. Briefly, urine samples from the local biobank were stored frozen at −80 C, thawed on ice, then centrifugated at 1500 relative centrifugal force (g) for 2 minutes, as to pellet all debris which might cause microfluidic channel obstruction. For Single Plex cartridge loading, 50 μL of each diluted urine supernatant sample (1:1 in Sample Diluent) or quality control was added to the wells, as well as 1 mL of Wash Buffer in the dedicated inlet. The automated Ella® immunoassay protocol was then initiated, including automated three times sampling of each well to give results in triplicate. Measurement of creatinine was performed in the same urine samples using the Creatinine Parameter Assay Kit (Bio-Techne).

Sample Preparation Study

As part of the local routine biobanking, urine samples are collected and processed as follows: samples are kept at room temperature (RT) until centrifuged at 3300 rpm for 20 minutes at 4° C. within 3 hours of collection. The supernatant is collected, split into two 15 mL tubes and stored with or without protease inhibitors (cOmplete™, Roche Diagnostics, Meylan, France) at −80° C. In this Sample Preparation Study, both aliquots of 25 urine samples were thawed on ice and urinary chemokines were quantified by Ella® technique in a single batch. Chemokine levels in each sample were compared according to the addition or not of protease inhibitors during sample preparation.

Storage Study

Fresh urine samples (N=5) were prospectively collected from hospitalized kidney transplant recipients presenting with a condition usually associated with high urinary chemokines levels (i.e. acute rejection, BKV replication or bacterial urinary tract infection), and split into 7 aliquots subjected to various procedures to produce 7 samples from each. A first aliquot (standard tube) was immediately centrifuged and stored without protease inhibitors at −80° C. The other aliquots were left for 24/48/72H, respectively at 4° or at room temperature (RT). Samples were centrifuged immediately before storage without protease inhibitors, and kept at −80° C. until analysis by Ella® technique in a single batch. Chemokine concentrations in each sample type were compared to those from the corresponding standard tube (see details in Statistical analysis).

Freeze/Thaw Cycles Study

Urine samples (N=5) were aliquoted into 5 tubes and stored at −80° C. without protease inhibitors until further analysis. Samples were thawed on ice during 2 h and frozen again at −80° C. on consecutive days. This procedure was performed in respective aliquots once (T1), twice (T2), three (T3), four (T4) or five (T5) times. Samples were kept at −80° C. until analysis by Ella® technique in a single batch. Chemokine concentrations in T2/T3/T4/T5 sample were compared to those from the matching T1 tube (see details in Statistical analysis). In this study, T1 corresponding to one freeze-thaw cycle is considered as the reference method and mirrors clinical use where samples are usually stored until filling-up the assay-plate.

Linearity

Urine samples (N=10) with a previous chemokine quantification were chosen to encounter for a broad range of CXCL9 and CXCL10 concentration, and diluted 1:2, 1:4, 1:8 and 1:16 in Sample Diluent (SD13, Simple Plex™, Bio-Techne). All diluted samples were assayed within a single run. Linearity was assessed by mean of the coefficient of variation (CV) with 1:2 dilution taken as the reference sample, and by Spearman correlation tests.

Accuracy and Limit of Quantification

Accuracy on urine samples of Ella® internal calibration curve was assessed by using recombinant Human CXCL9 form the Human CXCL9/MIG DuoSet ELISA kit (Bio-Techne). Urinary CXCL9 and CXCL10 levels were measured in urine samples (N=10) collected from kidney living-donors (KD) prior to kidney donation. Samples from various age, male and female KD were selected to bring diversity (data not shown) and pooled together. Recombinant Human CXCL9 standard (lot) was reconstituted with 0.5 mL of Reagent Diluent (RD, Catalog #DY995, Bio-Techne). Three different diluents were used: Sample Diluent (2:3 dilution of RD 1X in PBS, 0.025% Tween-20) for ELISA quantification, Sample Diluent SD13 (Simple Plex™, Bio-Techne) for Ella® quantification and the pooled urine samples from KD. For each of the diluent, eleven point standard curve using 2-fold serial dilutions was prepared. The resultant samples were quantified both by ELISA and Ella® technique in a single batch. Percent recovery was calculated for each of the 11 points, based on the found concentration and the theoretical concentration.

Within- and Between-Run Precision

Within-run (intra-assay) precision was assessed on urine samples (N=5) quantified twice on a same CXCL9 or CXCL10 cartridge. Between-run (inter-assay) variation was assessed for CXCL9 and CXCL10 on urine samples (N=5) quantified by two different technicians on different days with cartridges from the same lot. For CXCL9, intermediate precision was further refined in a larger number of samples (N=32) to assess technician-to-technician and day-to-day variations in high, mid and low CXCL9 concentration samples. Precision was expressed as CV.

Clinical Accuracy Study

To evaluate the diagnostic performances of urinary CXCL9 and 10 quantified by Ella® as compared to the reference ELISA method, we used a previously published cohort (Rabant M, J Am Soc Nephrol 2015). Among the 281 urine samples included in the original work, enough material was available for 275 of them, comprising 78 acute rejection samples. CXCL9 and CXL10 levels were measured in those 275 samples using the microfluidic Simple Plex cartridges Ella®. Accuracy was assessed by mean of an Area Under the recipient operating Curve (AUC). AUCs were then individually calculated for both chemokines, as raw data or normalized by urinary creatinine (CXCL9, CXCL10, CXCL9:cr and CXCL10:cr). The six unavailable urine samples belonged to patients within the “no rejection” group. In order to compare AUC's derived from the same cases, ELISA AUCs from the initial Rabant et al's work were calculated again by excluding the same six patients. AUCs were compared using the DeLong test.

8-Parameter Chemokine Model Coefficient Adjustment

To investigate how the modification in urinary chemokine quantification method might influence the performance of the 8-parameter chemokine model (Tinel C, Am J Transplant 2020), we used the same samples as the previously published cohort. Material was available for all 371 urine samples used in the building of the model, including 91 acute rejection samples. CXCL9 and CXL10 levels were measured in those 371 samples using the microfluidic Simple Plex cartridges Ella®. The logistic regression built 8-parameter model was then trained using urine chemokine levels by Ella® technology, and performance was assessed as AUCs.

Statistical Analyses

Changes in concentrations of chemokines over time, temperature or freeze-thaw cycles were analyzed using one-way repeated measures analysis of variance (RM-ANOVA) followed by Sidak's multiple comparisons tests. AUC's were compared with the Delong's test.

Statistical analyses were performed using Graphpad Prism version 9.0.1 (GraphPad Software, San Diego, USA) and with R software (R Development Core Team, R version 4.0.3 and R studio version 1.3.1093).

Results

Effects of Preanalytical Sample Processing on Urine Chemokines Assessment

If urinary chemokines are to be used for routine surveillance of KTRs, urine sample collection and processing have to be optimized to fit hospital's constraints (data not shown, Sample collection & storage). In research, protease inhibitors (PI) are usually added after urine supernatant collection to prevent protein degradation upon storage. However, this additional step during sample preparation is time consuming, costly, and might prevent consistent practice between centers. Thus valid information about necessity of PI addition to prevent CXCL9 and CXCL10 degradation is essential. Chemokine levels of 25 urine samples were compared according to the addition or not of PI during sample preparation. Median time from sample collection to quantification was 147 days [IQR: 127-184]. A nearly perfect correlation for CXCL9 was assessed (Spearman r=0.98 [95% CI: 0.96-0.99], P<0.0001), with a mean with/without PI ratio=1. For CXCL10, a high degree of correlation was also found (Spearman r=0.96 [95% CI: 0.92-0.98], P<0.0001). However, levels in aliquots with PI were slightly higher than those without PI (mean with/without PI ratio=1.1), suggesting that CXCL10 protein might be more fragile leading to a possible degradation over time in the absence of PI.

Effects of Processing Delay and Storage Conditions on Urine Chemokines Assessment

If routinely implemented, urine chemokine assessment might not be available in each single hospital and shipment to a centralized reference center might be considered. Besides, freezing a urine specimen prior to centrifugation may cause cell lysis upon thawing, allowing cellular cytoplasmic protein to contaminate the urine specimen. In research, an early centrifugation is thus usually performed to pellet cells, but it requires an available technician and a dedicated equipment. Thus, we investigated the influence of time and storage conditions on chemokine quantification. Fresh urine samples from 5 patients were kept at 4° C. or RT for respectively 24H, 48H or 72H. Centrifugation to pellet urine cells and collect urine supernatant was performed immediately before −80° C. storage. Within-person stability of CXCL9 and CXCL10 was assessed by RM-ANOVA, which showed no significant difference over time for samples kept at 4° C. (P=0.26 and P=0.79, data not shown) or at RT (P=0.13 and P=0.51, (data not shown). Up to 72H at RT, mean intra-patient CV did not exceed 20% for CXCL9 (4° C., CV=19.28%; RT, CV=17.51%) and 15% for CXCL10 (4° C., CV=13.12%; RT, CV=10.41%). Percent change in chemokine level was consistently positive across conditions, indicating a minor increase in chemokine levels upon time. Main variation happened within the first 24 h, suggesting cell lysis from urine cell pellet with adds-on from intracellular chemokines. Overall, mean percent change of each chemokine level remained low (<50%), indicating global stability of urine chemokine quantification and no major impact of processing delay in samples kept at 4° C. or RT and up to 72H.

Effects of Repeated Freeze—Thaw Cycles on Urine Chemokines Assessment

Nowadays targeted laboratory diagnostics results in a reduction of initial blood or urine sampling and additional laboratory test requests might be performed from the same sample (chemokines, proteinuria, urine creatinine . . . ). Besides, for organizational choice within the hospital laboratory, CXCL9 and CXCL10 levels might not be quantified on the same day. Finally, in case of a doubtful result, a second quantification of the same sample might be necessary. However, repeated freezing and thawing of samples may influence the stability of urine constituents. Results of analyses performed in urine samples exposed to repeated freeze-thaw cycles might therefore differ from analyses performed in fresh, or only once thawed samples. We thus investigated the influence of repeated freeze—thaw cycles on both chemokines quantified in 5 urine samples (data not shown). In comparison to samples thawed only once (T1), up to 4 additional cycles (T2-T5) did not significantly change within-patient chemokine levels (RM-ANOVA: CXCL9 P=0.79, CXCL10 P=0.26). The percentage change and CV in CXCL9/CXCL10 concentration were calculated for each refrozen sample in comparison to the baseline T1 sample. With mean CV of 10.63% (CXCL9) and 18.90% (CXCL10), both assays were found to meet the FDA acceptance criteria for bioanalytical method validation (<20%, https://www.fda.gov/). Percentage change remained low for both chemokines and was consistently negative for CXCL10, suggesting again a more fragile protein, with possible degradation over repeated freeze-thaw cycles.

Evaluation of Assay Preparation During Ella® Workflow

To investigate the feasibility of clinical implementation of Ella® quantification for urine proteins, we compared ELISA and Ella® workflow, from sample thaw to render of the results. Upon assay preparation, Ella® appeared superior to conventional ELISA with no plate coating and no tedious reagent preparation. Sample preparation only included thawing and an additional centrifugation step as to pellet all debris which might cause GNR obstruction (data not shown, Assay preparation). Ella® procedure further included a dilution step as for ELISA and a simple one-step sample deposition within the cartridge prior to running the assay (20 minutes). Once launched, time to result is approximately 70 minutes. Altogether, the estimated Ella® assay procedure is 1 h30 as compared to 7 h for a conventional ELISA (let alone antibody coating the day before for CXCL9/MIG DuoSet ELISA kit). Of importance, the Ella® assay only requires 354, of urine supernatant to generate triplicate data, suggesting the possibility of quantifying more analytes from a single precious sample. Finally, most recent Ella® cartridges offer the possibility of a combined CXCL9/CXCL10 quantification, providing urine levels for both chemokines and for up to 32 samples (including low and high quality controls) within a fast turnaround time.

How promising preanalytical studies and assay preparation are, one should not forget that Ella® platform has been tested on various body fluids including urine, but that specific CXCL9/10 cartridges have not been validated on human urine samples. Considering the wide range of pH and urine specific gravity, and that urine complex matrix may hinder immunologic testing, we run an in-house validation of all aspects of analytical performance of the assay (data not shown, Chemokine quantification).

Linearity and Range of Measurement

Ella® cartridges are provided with an internal calibration curve, i.e. a relationship between fluorescence and known concentrations of the analyte. But a calibration curve should be prepared in the same biological matrix as the sample. First we investigated the ability of the assay to produce results that are directly proportional to the concentration of analyte in the urine sample. Linearity was assessed from 10 urine samples with a broad range of chemokine values from previous measurement, subjected to serial dilution (1:2, 1:4, 1:8 and 1:16). A high repeatability for each sample was assessed with mean intra-patient CV of 10.2% for CXCL9 and 9.3% for CXCL10 (data not shown). From a Spearman correlation analysis between each dilution factor, all r values were ≥0.98 (data not shown). For CXCL10, linearity was confirmed within the complete range given by the manufacturer (dilution-corrected range 1.2-1840 pg/mL). For CXCL9 (manufacturer's range: 39.8-60,800 pg/mL), linearity was found reliable between 100 and 10,000 pg/mL, but was less clear for extreme values. Hence for sample 10 (data not shown), CXCL9 deviated from 8732 pg/mL (1:8 dilution) to 17464 pg/mL (1:16 dilution, CV=47.1%, % change=100). To further define CXCL9 lower and upper limits of quantification (LLOQ-ULOQ) on urine, we used recombinant CXCL9 serially diluted (1:2) into Sample Diluent or into pooled urine from healthy kidney donors, all with undetectable CXCL9 levels (data not shown). Recovery at 11 different spiked concentrations showed less reliable CXCL9 assessment below (expected value) 31.3 pg/mL and above 4000 pg/mL (data not shown). Overall, our linearity and recovery data support the following LLOQ and ULOQ on urine sample: 39.8-4000 pg/mL (CXCL9) and 0.6-920 pg/mL (CXCL10).

Within- and Between-Run Precision

For both assays, precision was assessed on 5 urine samples quantified twice on the same cartridge (intra-assay precision), or quantified twice by different technicians on different days (inter-assay precision). The intra-assay and inter-assay CVs were 4.7% and 15.3%, respectively for CXCL9, and 2.6% and 16.6%, respectively for CXCL10 (data not shown). For CXCL9, intermediate precision was further refined in a larger number of samples (N=32) to assess technician-to-technician and day-to-day variations in high, mid and low CXCL9 concentration samples. Under the same set of conditions and within a short interval of time, repeatability ranged from 3.8% (mid CXCL9) to 11.6% (low CXCL9). When investigating the random error introduced by factors like specific technicians, between-run variation was also found acceptable with CV ranging from 9.6% (low CXCL9) to 15.3% (high CXCL9). The inter-assay CV for all 37 tested samples averaged 10.3%.

Clinical Accuracy Study

To evaluate the clinical performances of uCXCL9 and uCXCL10 quantified by Ella®, 600 samples belonging to 2 previously published cohorts (Cohort A and B) were quantified again using the Ella® method. Results shows a high degree of correlation between uCXCL9 and uCXCL10 measurements by Ella® and by the reference ELISA method (P<0.0001). More specifically, assessments from the 2 methods were compared using Bland-Altman test (data not shown). For uCXCL10, both methods provided very superimposable values, with uCXCL10 Ella®/ELISA ratio mostly distributed around 1 (Bias=0.91; 95% CI, −0.81:2.62). For uCXCL9, though highly correlated, numerical values were always found higher when quantified by Ella® in comparison to ELISA (Bias=2.80; 95% CI, −3.5:9.07). Though unexpected, these results (combined with the previous recovery study using recombinant CXCL9) suggest that Ella® might provide a more accurate numerical quantification for CXCL9, than ELISA did. Besides, we compared AUC values in cohort A and B, from urine chemokines measured with ELISA or Ella® method. In cohort A, AUCs were generated for multiple endpoints (acute rejection, ABMR, TCMR . . . ), for CXCL9 or CXCL10, as raw data or normalized by urine creatinine. CXCL9 AUCs were improved when measured by Ella®, while CXCL10 AUCs were slightly degradated (data not shown). Despite these minor variations, global diagnostic accuracy of Ella® was found similar to that of ELISA. Finally, we previously established a model of acute rejection risk using urine chemokines and their confounding factors. To move this model forward to clinical use, we aim at training the model on Ella®-generated data, rather than on ELISA data. The derivation cohort (N=372) was quantified again using Ella®. Overall, when trained on Ella® data, diagnostic accuracy of the 8-parameter model remained unchanged (DeLong's P-value=0.44, FIG. 12).

The Web Application Calculator for Assessment of Acute Rejection Risk Using Urine Chemokines

The model was first derived in KTR from Necker Hospital, and validated in an external single-center cohort and in a prospective multicenter unselected cohort. All samples from these 3 cohorts have since been quantified again by Ella® method, enabling to train and validate the model on Ella® data. The resulting model reached an AUC of 0.84 (CI: 0.80-0.89) for any rejection diagnosis. For clinical assessment of a patient's risk for acute rejection, and either prompt the decision in performing a biopsy, either argue for avoiding an unnecessary biopsy, we have built a web application calculator www.optim.care.demo/. Transplant specialists may now easily enter their patients clinical data (age, gender), serum lab tests (creatinine, DSA and BKV viral load) and urine lab tests (creatinine, uCXCL9 and uCXCL10 levels), and rapidly get an accurate risk prediction (data not shown, right Panel). For ease of use, lab test results may be entered in various units with build-in conversion calculation. Health Care Professionals may register their unit preference for future use as well as a create a patient's profile, allowing time intervals between score to be graphically displayed and listed (data not shown). Finally, for flexibility missing data can be imputed by last recorded data (e.g. no recent DSA assessment but patient was always DSA negative), or by mean imputation (e.g. missing BKV viral load imputed by mean viral load).

Optimized Integrative Model Using Urinary Chemokines for Noninvasive Diagnosis of Acute Allograft Rejection

Our optimized integrative model was developed under R environment, using a multivariable logistic regression to assess the relationship between the outcome “acute rejection” and several predictor variables (Table 9). CXCL9 and CXCL10 protein expression in urine supernatant were normalized to the urinary creatinine (cr) level through determination of the CXCL9/cr and CXCL10/cr ratio (nanograms of protein per millimole of urinary creatinine). CXCL9/cr and CXCL10/cr were transformed with natural logarithm.

In conclusion, the current study has identified and validated an improved method for the quantification of urine CXCL9 and CXCL10 for clinical use. Firstly, Ella® assay accurately measured both chemokines from urine samples. Secondly, a great simplification of preanalytical sample processing was reached with stability up to 72H at room temperature without any prior centrifugation or adjunction of preservative after urine collection. Thirdly, the fully-automated assay provides unprecedented rapid assessment, fitting the clinical expectations in render of the results. Fourthly, training our previously published model on Ella® data enabled to validate diagnostic accuracy and to develop an online available web application. Given these, urine CXCL9 and CXCL10 now display all characteristics for moving from research to clinical surveillance acute rejection in KTRs: time has come to put words into actions.

TABLE 1
Clinical, histological and biological characteristics
at the time of allograft biopsy

| Variables | N = 391 | |
|---|---|---|
| Time after transplantation (mo). median (IQR) | 8 | (33) |
| Indication of biopsy | | |
| Screening biopsy. n (%) | 46 | (11.8) |
| Clinically indicated biopsy. n (%) | 345 | (88.2) |
| Rise in serum creatinine. n (%) | 214 | (54.7) |
| Proteinuria. n (%) | 32 | (8.2) |
| De novo DSAs. n (%) | 10 | (2.6) |
| Control after rejection. n (%) | 46 | (11.8) |
| BKV viremia. n (%) | 43 | (11.0) |
| Other. n (%) | 1 | (0.3) |
| Pathologic primary diagnosis | | |
| ABMR. n (%) | 64 | (16.4) |
| TCMR. n (%) | 17 | (4.3) |
| Mixed rejection. n (%) | 14 | (3.6) |
| BKVN. n (%) | 23 | (5.9) |
| IF/TA. n (%) | 154 | (39.4) |
| Acute tubular injury. n (%) | 11 | (2.8) |
| Recurrent disease. n (%) | 9 | (2.3) |
| Normal. n (%) | 21 | (5.4) |
| Other^{a}. n (%) | 78 | (20.0) |
| Laboratory test results at the time of biopsy | | |
| Serum | | |
| Creatinine (μmol/L). mean ± SD | 188 ± 102 | |
| DSAs. n (%)^{b} | 155 | (40.6) |
| Detectable BKV viremia. n (%) | 62 | (15.9) |
| Viral load (Log₁₀ copies/mL). median (IQR) | 3.5 | (1.8) |
| Urine | | |
| Proteinuria/creatininuria ratio (g/g). mean ± SD | 0.8 ± 1.7 | |
| Bacteriuria (≥10³/mL) and | 39 | (10.0) |
| leukocyturia (≥10⁴/mL). n (%) | | |

Abbreviations: ABMR, antibody-mediated rejection; BKVN, BK-virus nephropathy; DSAs, donor-specific antibodies; IF/TA, interstitial fibrosis/tubular atrophy; SD, standard deviation; TCMR, T-cell-mediated rejection. Borderline rejection lesions were classified among normal biopsies (N=14). Blood BKV viral load is expressed as the number of copies (log10) per mL of plasma. a Nonspecific lesions including calcineurin inhibitor toxicity. b Data not available for 9 cases.

TABLE 2
Association between urinary chemokines and clinical and laboratory data

| | LnCXCL9/cr | | | LnCXCL10/cr | | |
|---|---|---|---|---|---|---|
| | β | 95% Cl | P | β | 95% Cl | P |
| Demographic variables | | | | | | |
| Recipient age | 0.002 | −0.01-0.01 | 0.695 | −0.001 | −0.01-0.01 | 0.906 |
| Recipient sex: | | | | | | |
| Male | 0.237 | −0.14-0.61 | 0.213 | −0.154 | −0.56-0.25 | 0.459 |
| Donor age | 0.001 | −0.01-0.01 | 0.789 | 0.005 | −0.01-0.02 | 0.412 |
| Donor type: | | | | | | |
| Living donor | −0.224 | −0.63-0.19 | 0.284 | −0.158 | −0.61-0.29 | 0.491 |
| Preformed DSAs | −0.102 | −0.47-0.26 | 0.581 | 0.201 | −0.19-0.61 | 0.305 |
| At the time of transplantation | | | | | | |
| DGF | 0.316 | −0.05-0.68 | 0.089 | 0.148 | −0.25-0.55 | 0.469 |
| Induction therapy: | | | | | | |
| Thymoglobuline ® | −0.168 | −0.54-0.2 | 0.376 | 0.102 | −0.31-0.51 | 0.623 |
| Time from transplantation to biopsy | 0.003 | 0-0.01 | 0.158 | 0.003 | 0-0.01 | 0.227 |
| Laboratory test | | | | | | |
| results at the time of biopsy | | | | | | |
| DSAs | 0.405 | 0.04-0.77 | 0.032 | 0.575 | 0.17-0.98 | 0.006 |
| Serum creatinine | 0.004 | 0-0.01 | <0.001 | 0.004 | 0-0.01 | <0.001 |
| Proteinuria/creatininuria ratio | 0.014 | 0.01-0.02 | <0.001 | 0.156 | 0.01-0.02 | <0.001 |
| Histological variables | | | | | | |
| ABMR | 1.006 | 0.56-1.45 | <0.001 | 1.138 | 0.65-1.62 | <0.001 |
| TCMR | 2.037 | 1.40-2.67 | <0.001 | 1.610 | 0.90-2.32 | <0.001 |
| Acute rejection (ABMR, | 1.194 | 0.79-1.60 | <0.001 | 1.199 | 0.75-1.65 | <0.001 |
| TCMR, mixed) | | | | | | |
| BKV infection | | | | | | |
| Detectable BKV viremia | 1.215 | 0.73-1.70 | <0.001 | 1.620 | 1.10-2.14 | <0.001 |
| BKVN | 1.163 | 0.40-1.93 | 0.003 | 1.477 | 0.64-2.31 | <0.001 |
| Bacterial UTI | | | | | | |
| Leukocyturia (≥10⁴/mL) | 1.116 | 0.71-1.53 | <0.001 | 1.142 | 0.69-1.59 | <0.001 |
| Bacteriuria (≥10³/mL) and | 0.946 | 0.35-1.55 | 0.002 | 1.210 | 0.55-1.86 | <0.001 |
| leukocyturia (≥10⁴/mL) | | | | | | |

Abbreviations: ABMR, antibody-mediated rejection;
BKVN, BK-virus nephropathy;
CPU, colony-forming units;
Cl, confidence interval;
cr, urinary creatinine;
DGF, delayed graft function;
DSAs, donor-specific antibodies;
TCMR, T-cell-mediated rejection;
UTI, urinary tract infection (bacteriuria [≥10³CFU/mL] and leukogyturia [≥10⁴/ml]).

TABLE 3
Multivariable model for the diagnosis of acute allograft rejection

| | Adjusted OR | 95% CI | P-value |
|---|---|---|---|
| Clinical variables at the time of transplantation | | | |
| Recipient sex (F) | 2.43 | 1.34-4.47 | 0.004 |
| Recipient age | 0.97 | 0.95-0.99 | 0.005 |
| Laboratory test results at the time of biopsy | | | |
| eGFR (MDRD): | | | |
| 30-59 mL/min/1.73 m² | 4.34 | 1.16-22.98 | 0.049 |
| 15-29 mL/min/1.73 m² | 8.17 | 2.05-45.39 | 0.007 |
| <15 mL/min/1.73 m² | 12.27 | 2.29-84.47 | 0.006 |
| DSA score: | | | |
| 500 ≤ MFI <1000 | 2.02 | 0.83-4.75 | 0.113 |
| 1000 ≤ MFI <3000 | 4.08 | 1.86-9.08 | 4.8E−04 |
| 3000 ≤ MFI | 4.15 | 1.81-9.60 | 0.001 |
| Confounding factors | | | |
| Blood BKV viral load (upper quartile, ≥4.3 log) | 0.08 | 0.00-0.51 | 0.026 |
| UTI | 0.27 | 0.08-0.74 | 0.016 |
| Chemokines | | | |
| LnCXCL9/cr | 1.41 | 1.16-1.73 | 0.001 |
| LnCXCL10/cr | 1.26 | 1.04-1.55 | 0.021 |

TABLE 4
Univariate and multivariable logistic regression analysis

| | Univariate analysis | | | Multivariable analysis | | |
|---|---|---|---|---|---|---|
| | OR | 95% Cl | P-value | Adjusted OR | 95% Cl | P-value |
| Clinical variables at the time | | | | | | |
| of transplantation | | | | | | |
| Retransplantation | 1.46 | 0.82-2.52 | 0.188 | 1.28 | 0.63-2.55 | 0.483 |
| Recipient sex (F) | 2.19 | 1.36-3.56 | 0.001 | 2.48 | 1.36-4.62 | 0.003 |
| Recipient age | 0.97 | 0.96-0.99 | 2.4E−04 | 0.97 | 0.95-0.99 | 0.005 |
| Donor type (deceased donor) | 1.01 | 0.60-1.75 | 0.972 | | | |
| Time from transplantation | 1.08 | 0.66-1.75 | 0.761 | | | |
| Induction therapy (basiliximab) | 1.18 | 0.741.91 | 0.488 | | | |
| Biological variables at the | | | | | | |
| time of biopsy | | | | | | |
| eGFR (MDRD): | | | | | | |
| 30-59 mL/min | 2.74 | 0.92-11.80 | 0.109 | 4.83 | 1.21-28.44 | 0.046 |
| 15-29 mL/min | 3.79 | 1.24-16.56 | 0.037 | 8.67 | 2.07-53.22 | 0.008 |
| <15 mL/min | 12.22 | 3.0-6.598 | 0.001 | 11.99 | 2.13-89 | 0.008 |
| Proteinuria/creatininuria ratio | 1.53 | 1.23-1.92 | 1.3E−04 | 1.25 | 0.94-1.66 | 0.126 |
| DSA score: | | | | | | |
| 500 ≤ MFI <1000 | 1.79 | 0.80-3.79 | 0.141 | 2.03 | 0.82-4.82 | 0.114 |
| 1000 ≤ MFI <3000 | 4.03 | 2.06-7.88 | 4.4E−05 | 3.86 | 1.74-8.67 | 0.001 |
| 3000 ≤ MFI | 8.44 | 4.30-16.97 | 1.0E−09 | 3.68 | 1.58-8.64 | 0.003 |
| Confounding factors | | | | | | |
| BKV viremia (1^{st} quartile, | 1.29 | 0.34-4.08 | 0.679 | 0.98 | 0.19-4.3 | 0.978 |
| 2.4≤ × <2.5 log) | | | | | | |
| BKV viremia (2^{nd} quartile, | 1.16 | 0.31-3.57 | 0.806 | 0.90 | 0.20-3.56 | 0.886 |
| 2.55≤ × <3.48 log) | | | | | | |
| BKV viremia (3^{rd} quartile, | 0.00 | 0.00-8.54 | 0.9807 | 0.00 | 0.00-1.12 | 0.986 |
| 3.48≤ × <4.3 log) | | | | | | |
| BKV viremia (upper | 0.19 | 0.01-0.98 | 0.114 | 0.09 | 0.00-0.59 | 0.035 |
| quartile, ≥4.3 log) | | | | | | |
| Significant UTI | 0.56 | 0.21-1.30 | 0.210 | 0.24 | 0.07-0.68 | 0.011 |
| Chemokines | | | | | | |
| LnCXCL9/cr | 1.41 | 1.24-1.62 | 2.7E−07 | 1.40 | 1.15-1.72 | 9.4E−04 |
| LnCXCL10/cr | 1.42 | 1.23-1.65 | 3.0E−06 | 1.24 | 1.02-1.52 | 3.3E−02 |

TABLE 5
β coefficent for each variable included in the optimized model

| | Variable | β coefficient |
|---|---|---|
| Intercept | x₀ | −2.75885 |
| Clinical variables at the time of transplantation | | |
| Recipient sex (F) | x₁ | 0.88720 |
| Recipient age | x₂ | −0.02971 |
| Biological variables at the time of biopsy | | |
| eGFR (MDRD): | x₃ | |
| 30-59 mL/min | | 1.46782 |
| 15-29 mL/min | | 2.10024 |
| <15 mL/min | | 2.50680 |
| DSA score: | x₄ | |
| 500 ≤ MFI <1000 | | 0.70202 |
| 1000 ≤ MFI <3000 | | 1.40666 |
| 3000 ≤ MFI | | 1.42259 |
| Confounding factors | | |
| Blood BKV viral load | x₅ | |
| 1^{st} quartile, 2.4 ≤ × <2.5 log | | −0.20666 |
| 2^{nd} quartile, 2.5 ≤ × <3.48 log | | −0.18699 |
| 3^{d} quartile, 3.48 ≤ × <4.3 log | | −16.94774 |
| Upper quartile, ≥4.3 log | | −2.56569 |
| Significant UTI | x₆ | −1.32241 |
| Chemokines | | |
| LnCXCL9/cr | x₇ | 0.34557 |
| LnCXCL10/cr | x₈ | 0.23292 |

Abbreviations: CI, confidence interval; cr, urinary creatinine; DSA, donor-specific antibody; eGFR, estimated glomerular filtration rate; F, female; MDRD, modification of diet in renal disease; MFI, mean fluorescence intensity; OR, odds ratio; UTI, urinary tract infection.

TABLE 6
Sample characteristics from the four non-overlapping groups in the cross-sectional study.

| | | | | | | |
|---|---|---|---|---|---|---|
| | All | | | | | |
| | samples | No BKV | Viruria | DNAemia | BKVN | |
| Variables | n = 474 | n = 262 | n = 135 | n = 52 | n = 25 | P-value |

| | | | | | | | | | | | |
|---|---|---|---|---|---|---|---|---|---|---|---|
| Time after transplantation (mo), median (IQR) | 11 | (34) | 11 | (46) | 11 | (28) | 9 | (19) | 7 | (17) | 0.97 |

Indication for biopsy

| Screening biopsy, n (%) | 58 | (12.2) | 31 | (11.8) | 24 | (17.8) | 3 | (5.8) | 0 | | <0.05 |
|---|---|---|---|---|---|---|---|---|---|---|---|
| Clinically indicated biopsy, n (%) | 416 | (87.8) | 231 | (88.2) | 111 | (82.2) | 49 | (94.2) | 25 | (100) | |
| Allograft dysfunction, n (%) | 259 | (62.3) | 163 | (70.6) | 70 | (63.1)) | 19 | (38.8) | 7 | (28.0) | <0.0001 |
| Proteinuria, n (%) | 39 | (9.4) | 27 | (11.7) | 11 | (9.9) | 0 | | 1 | (4.0) | 0.06 |
| De novo DSAs, n (%) | 12 | (2.9) | 7 | (3.0) | 5 | (4.5) | 0 | | 0 | | 0.35 |
| BKV DNAemia, n (%) | 47 | (11.3) | 3 | (1.3) | 0 | | 28 | (57.1) | 16 | (64.0) | <0.0001 |
| Other, n (%) | 59 | (14.2) | 31 | (13.4) | 25 | (22.5) | 2 | (4.1) | 1 | (4.0) | <0.01 |

Pathologic primary diagnosis (except BKVN)

| | | | | | | | | | | | |
|---|---|---|---|---|---|---|---|---|---|---|---|
| Inadequate, n (%) | 32 | (6.8) | 18 | (6.9) | 8 | (5.9) | 6 | (11.5) | 1 | (4.0) | 0.52 |
| Acute rejection, n (%) | 102 | (21.5) | 65 | (24.8) | 28 | (20.7) | 9 | (17.3) | 1 | (4.0) | 0.08 |

| | | | | | | | | | | |
|---|---|---|---|---|---|---|---|---|---|---|
| Normal, n (%) | 23 | (4.9) | 13 | (5.0) | 7 | (5.2) | 3 | (5.8) | NA | 0.97 |
| Other lesions, n (%)^{a} | 292 | (61.6) | 166 | (63.4) | 92 | (68.1) | 34 | (65.4) | NA | 0.64 |

BKV infection characteristics

| Detectable BKV DNAemia, n (%) | 76 | (16.0) | NA | NA | | 52 | (100) | 24 | (96.0) | 0.71 |
|---|---|---|---|---|---|---|---|---|---|---|
| Viral load (Log₁₀ copies/mL), | 3.32 | (3.1) | NA | NA | | 2.68 | (1.2) | 3.96 | (1.9) | <0.001 |
| median (IQR) | | | | | | | | | | |
| Detectable BKV viruria, n (%) | 204 | (43.0) | NA | 135 | (100) | 45 | (86.5) | 24 | (96.0) | <0.0001 |

| | | | | | | |
|---|---|---|---|---|---|---|
| Viral load (copies/ng), median (IQR) | 7.2E+05 | NA | 7.3E+04 | 6.8E+08 | 3.6E+09 | <0.0001 |
| | (6.8E+08) | | (2.2E+06) | (3.2E+09) | (1.2E+10) | |

| | | | | | | | | |
|---|---|---|---|---|---|---|---|---|
| BKVN, n (%) | 25 | (5.3) | NA | NA | NA | 25 | (100) | NA |

| | | | | | | |
|---|---|---|---|---|---|---|
| Laboratory test results at the time of biopsy | | | | | | |
| Serum creatinine (μmol/L), mean ± SD | 185 ± 99 | 194 ± 116 | 176 ± 78 | 167 ± 53 | 186 ± 71 | 0.61 |

| | | | | | | | | | | | |
|---|---|---|---|---|---|---|---|---|---|---|---|
| DSAs, n (%)^{b} | 184 | (40.5) | 105 | (42.0) | 51 | (38.9) | 18 | (37.5) | 10 | (40.0) | 0.91 |

| | | | | | | |
|---|---|---|---|---|---|---|
| Proteinuria/creatininuria ratio (g/g), mean ± SD | 0.85 ± 1.6 | 1.0 ± 1.9 | 0.71 ± 1.4 | 0.49 ± 0.7 | 0.34 ± 0.6 | <0.05 |

| | | | | | | | | | | | |
|---|---|---|---|---|---|---|---|---|---|---|---|
| Bacteriuria (≥10⁵/mL) and leukocyturia (≥10⁴/mL)^{c}, | 43 | (10.3) | 29 | (12.6) | 6 | (5.1) | 6 | (12.2) | 2 | (8.7) | 0.17 |
| n (%) | | | | | | | | | | | |

Abbreviations: BKVN, BKV-associated nephropathy;
DSAs, donor-specific antibodies;
IF/TA, interstitial fibrosis/tubular atrophy;
IQR, interquartile range;
SD, standard deviation.
^{a}Including calcineurin inhibitor toxicity, IF/TA and recurrent disease.
^{b}Data not available (NA) for 20 patients.
^{c}NA for 55 patients.

TABLE 7
Determinants of worsening postbiopsy allograft function, as assessed by the time to
reach 50% eGFR decline, by univariate and multivariate death-censored Cox analyses.

| | | Univariate | Univariate | Multivariate | Multivariate |
|---|---|---|---|---|---|
| Variable category | Explicative variables | HR (95% CI) | P-value | HR (95% CI) | P-value |
| Biological data | Serum creatinine | 1.56 (0.45-5.36) | 0.4806 | | |
| | DSAs at the time of biopsy | 0.81 (0.33-2.01) | 0.6550 | | |
| | Proteinuria/creatininuria ratio | 1.60 (0.94-2.73) | 0.0826 | 1.55 (0.90-2.68) | 0.1153 |
| | Blood BKV viral load | 2.88 (0.63-13.13) | 0.1707 | 2.01 (0.38-10.66) | 0.4142 |
| | Urine BKV viral load | 0.96 (0.87-1.06) | 0.3983 | | |
| Histological grading | i Banff elementary lesion | 0.57 (0.20-1.57) | 0.2744 | | |
| | t Banff elementary lesion | 1.17 (0.87-1.57) | 0.3116 | | |
| | ci Banff elementary lesion | 1.14 (0.77-1.70) | 0.5090 | | |
| | ct Banff elementary lesion | 1.10 (0.74-1.64) | 0.6329 | | |
| | ti Banff score | 0.93 (0.61-1.42) | 0.7528 | | |
| | i-IFTA Banff score | 1.00 (0.69-1.45) | 0.9947 | | |
| | MVI score | 1.28 (0.44-3.77) | 0.6518 | | |
| | BKVN | 0.98 (0.38-2.49) | 0.9610 | | |
| Urinary biomarker | uCXCL10/cr | 1.65 (1.08-2.51) | 0.0193 | 1.52 (1.00-2.30) | 0.0473 |

Abbreviations: BKVN, BKV-associated nephropathy; CI, confidence interval; ci, interstitial fibrosis; cr, urinary creatinine; ct, tubular atrophy; DSAs, donor-specific antibodies; eGFR, estimated glomerular filtration rate; HR, hazard ratio; I, interstitial infiltrate; i-IFTA, inflammation within areas of interstitial fibrosis and tubular atrophy; MVI, microvascular inflammation; t, tubulitis; ti, total inflammation.

Variables with a P-value <0.2 in the univariate Cox model were further entered into multivariate (death censored) Cox analysis. MVI is defined by the sum of the glomerulitis and peritubular capillaritis scores.

TABLE 8
BKV infection characteristics in the longitudinal study, according to urinary CXCL10 at first DNAemia.
According to urinary CXCL10 at first DNAemia

| | All patients | | Low CKCL10 | | High CXCL10 | | |
|---|---|---|---|---|---|---|---|
| Variables | N = 60* | | N = 22 | | N = 24 | | P value |
| Characteristics at first DNAemia | | | | | | | |
| Time from transplantation (mo), | 5.0 | (7.3) | 4.8 | (7.5) | 4.1 | (3.7) | 0.24 |
| median (IQR) | | | | | | | |
| eGFR (MDRD) (mL/min), median (IQR) | 44.2 | (21.7) | 46.7 | (17.2) | 41.5 | (24.6) | 0.18 |
| BKV viral load (Log₁₀ copies/mL), | 3.0 | (0.8) | 2.8 | (0.5) | 3.2 | (0.8) | 0.25 |
| median (IQR) | | | | | | | |
| Urinary CXCL10 (ng/mmol), | 13.6 | (11.1) | 7.6 | (5.7) | 18.9 | (9.6) | <0.0001 |
| median (IQR) | | | | | | | |
| Evolution during DNAemia | | | | | | | |
| Peak BKV viral load (Log₁₀copies/mL), | 3.55 | (1.3) | 3.6 | (1.2) | 3.6 | (1.3) | 0.89 |
| median (IQR) | | | | | | | |
| BKVN, n (%) | 8 | (13.3) | 3 | (13.6) | 4 | (16.7) | 1 |
| BKV clearance time (d), medion (IQR) | 105 | (146) | 131 | (205) | 132 | (148) | 0.49 |
| Peak urinary CXCL10, median (IQR) | 20.2 | (26.9) | 12.3 | (13.4) | 36.0 | (31.2) | <0.0001 |
| Urinary CXCL10 daily AUC during | 10.3 | (13.3) | 7.15 | (6.9) | 19.3 | (20.1) | <0.001 |
| DNAemiia (ng/mmol/d), median (IQR) | | | | | | | |

Abbreviations: AUC, area under the curve; BKVN, BKV-associated nephropathy; d, days; eGFR, estimated glomerular filtration rate; IQR, interquartile range; LOQ, limit of quantification; MDRD, modification of diet in renal disease; mo, months; qPCR, quantitative polymerase chain reaction; SD, standard deviation.

CXCL10 groups are defined according to uCXCL10/cr at first DNAemia: ≤12.86 ng/mmol (low) or >12.86 ng/mmol (high). First DNAemia is defined by the date of the first blood BKV qPCR LOQ. BKV clearance is defined by the time between first DNAemia and the 1st qPCR ≤LOQ with 2 consecutive concordant assessments. Urinary CXCL10 time-adjusted AUC (ng/mmol/d) is calculated from first DNAemia to BKV clearance, normalized by the number of days of BKV-DNAemia, and censored by the rejection date if any. *N=14 patients with no urinary CXCL10 assessments within 7 days from their 1st DNAemia could not be classified into the low/high-uCXCL10 groups.

TABLE 9
β coefficient for each variable included
in the optimized model (Ella ®)

| | Variable | β coefficient |
|---|---|---|
| Intercept | x₀ | −3.53296 |
| Clinical variables at the time of transplantation | | |
| Recipient sex (F) | x₁ | 0.92043 |
| Recipient age | x₂ | −0.02968 |
| Biological variables at the time of biopsy | | |
| eGFR(MDRD): | x₃ | |
| 30-59 mL/min/1.73 m² | | 1.23794 |
| 15-29 mL/min/1.73 m² | | 1.73871 |
| <15 mL/min/1.73 m² | | 1.96196 |
| DSA score: | x₄ | |
| 500 ≤ MFI <1000 | | 0.68086 |
| 1000 ≤ MFI <3000 | | 1.3356 |
| 3000 ≤ MFI | | 1.44792 |
| Confounding factors | | |
| Blood BKV viral load | x₅ | |
| 1^{st} quartile, 2.4 ≤ × <2.5 log | | −0.34922 |
| 2^{nd} quartile, 2.5 ≤ × <3.48 log | | −0.07594 |
| 3^{d} quartile, 3.48 ≤ × <4.3 log | | −16.81861 |
| Upper quartile, ≥4.3 log | | −2.56377 |
| Significant UTI | x₆ | −1.37589 |
| Chemokines | | |
| LnCXCL9/cr | x₇ | 0.6482 |
| LnCXCL10/cr | x₈ | 0.03591 |

### REFERENCES

Throughout this application, various references describe the state of the art to which this invention pertains. The disclosures of these references are hereby incorporated by reference into the present disclosure.
1. Mengel M. Deconstructing interstitial fibrosis and tubular atrophy: a step toward precision medicine in renal transplantation. Kidney Int. 2017; 92(3):553-555.
2. Fairchild R L, Suthanthiran M. Urine CXCL10/IP-10 Fingers Ongoing Antibody-Mediated Kidney Graft Rejection. J Am Soc Nephrol. 2015; 26(11):2607-2609.
3. Naesens M, Anglicheau D. Precision Transplant Medicine: Biomarkers to the Rescue. J Am Soc Nephrol. 2018; 29(1):24-34.
4. Jackson J A, Kim E J, Begley B, et al. Urinary chemokines CXCL9 and CXCL10 are noninvasive markers of renal allograft rejection and BK viral infection. Am J Transplant.
5. Hu H, Kwun J, Aizenstein B D, Knechtle S J. Noninvasive detection of acute and chronic injuries in human renal transplant by elevation of multiple cytokines/chemokines in urine. Transplantation. 2009; 87(12):1814-1820.
6. Hu M, Zhang G Y, Walters G, et al. Matching T-cell receptors identified in renal biopsies and urine at the time of acute rejection in pediatric renal transplant patients. Am J

Transplant. 2004; 4(11): 1859-1868.
7. Hirsch H H, Randhawa P, Practice ASTIDCo. BK polyomavirus in solid organ transplantation. Am J Transplant. 2013; 13 Suppl 4:179-188.
8. Allison P D. Multiple regression: a primer. Thousand Oaks, Calif.; London: Pine Forge Press; 1999.
9. Youden W J. Index for rating diagnostic tests. Cancer. 1950; 3(1):32-35.
10. DeLong E R, DeLong D M, Clarke-Pearson D L. Comparing the areas under two or more correlated receiver operating characteristic curves: a nonparametric approach. Biometrics. 1988; 44(3):837-845.
11. Lemeshow S, Hosmer D W, Jr. A review of goodness of fit statistics for use in the development of logistic regression models. Am J Epidemiol. 1982; 115(1):92-106.
12. Zhang Z, Rousson V, Lee W C, et al. Decision curve analysis: a technical note. Ann Transl Med. 2018; 6(15):308.
13. Vickers A J, Elkin E B. Decision curve analysis: a novel method for evaluating prediction models. Med Decis Making. 2006; 26(6):565-574.
14. Vickers A J, Van Calster B, Steyerberg E W. Net benefit approaches to the evaluation of prediction models, molecular markers, and diagnostic tests. BMJ. 2016; 352:i6.
15. Parasuraman R, Julian K, Practice ASTIDCo. Urinary tract infections in solid organ transplantation. Am J Transplant. 2013; 13 Suppl 4:327-336.
16. Hricik D E, Nickerson P, Formica R N, et al. Multicenter validation of urinary CXCL9 as a risk-stratifying biomarker for kidney transplant injury. Am J Transplant. 2013; 13(10):2634-2644.
17. Handschin J, Hirt-Minkowski P, Honger G, et al. Technical Considerations and Confounders for Urine CXCL10 Chemokine Measurement. Transplant Direct. 2020; 6(1):e519.
18. Brennan D C, Agha I, Bohl D L, et al. Incidence of BK with tacrolimus versus cyclosporine and impact of preemptive immunosuppression reduction. Am J Transplant. 2005; 5(3):582-594.
19. Koukoulaki M, Grispou E, Pistolas D, et al. Prospective monitoring of BK virus replication in renal transplant recipients. Transpl Infect Dis. 2009; 11(1):1-10.
20. Bressollette-Bodin C, Coste-Burel M, Hourmant M, Sebille V, Andre-Gamier E, Imbert-Marcille B M. A prospective longitudinal study of BK virus infection in 104 renal transplant recipients. Am J Transplant. 2005; 5(8):1926-1933.
21. Almeras C, Vetromile F, Garrigue V, Szwarc I, Foulongne V, Mourad G. Monthly screening for BK viremia is an effective strategy to prevent BK virus nephropathy in renal transplant recipients. Transpl Infect Dis. 2011; 13(2):101-108.
22. Hirsch H H, Brennan D C, Drachenberg C B, et al. Polyomavirus-associated nephropathy in renal transplantation: interdisciplinary analyses and recommendations. Transplantation. 2005; 79(10):1277-1286.
23. Mengel M. BK Virus Nephropathy Revisited. Am J Transplant. 2017; 17(8): 1972-1973.
24. Kariminik A, Dabiri S, Yaghobi R. Polyomavirus B K Induces Inflammation via Up-regulation of CXCL10 at Translation Levels in Renal Transplant Patients with Nephropathy. Inflammation. 2016; 39(4): 1514-1519.
25. Kariminik A, Yaghobi R, Dabiri S. Association of BK Virus Infection with CXCL11 Gene Expression and Protein Levels in Kidney Transplant Patients. Exp Clin Transplant. 2018; 16(1):50-54.
26. Weseslindtner L, Hedman L, Wang Y, et al. Longitudinal assessment of the CXCL10 blood and urine concentration in kidney transplant recipients with BK polyomavirus replication-a retrospective study. Transpl Int. 2020.
27. Drachenberg C B, Papadimitriou J C, Hirsch H H, et al. Histological patterns of polyomavirus nephropathy: correlation with graft outcome and viral load. Am J Transplant. 2004; 4(12):2082-2092.
28. Suthanthiran M, Schwartz J E, Ding R, et al. Urinary-cell mRNA profile and acute cellular rejection in kidney allografts. N Engl J Med. 2013; 369(1):20-31.
29. Roedder S, Sigdel T, Salomonis N, et al. The kSORT assay to detect renal transplant patients at high risk for acute rejection: results of the multicenter AART study. PLoS Med. 2014; 11(11):e1001759.
30. Schaub S, Nickerson P, Rush D, et al. Urinary CXCL9 and CXCL10 levels correlate with the extent of subclinical tubulitis. Am J Transplant. 2009; 9(6):1347-1353.
31. Hricik D E, Formica R N, Nickerson P, et al. Adverse Outcomes of Tacrolimus Withdrawal in Immune-Quiescent Kidney Transplant Recipients. J Am Soc Nephrol. 2015; 26(12):3114-3122.
32. Anglicheau D, Naesens M, Essig M, Gwinner W, Marquet P. Establishing Biomarkers in Transplant Medicine: A Critical Review of Current Approaches. Transplantation. 2016; 100(10):2024-2038.
33. Hirsch H H, Randhawa P S. BK polyomavirus in solid organ transplantation-Guidelines from the American Society of Transplantation Infectious Diseases Community of Practice. Clin Transplant [Internet]. 2019; 33(9). Available from: https://pubmed.ncbi.nlm.nih.gov/30859620/.
34. Tinel C, Devresse A, Vermorel A, Sauvaget V, Marx D, Avettand-Fenoel V, et al. Development and validation of an optimized integrative model using urinary chemokines for noninvasive diagnosis of acute allograft rejection. Am J Transplant [Internet]. 2020 April; Available from: http://www.ncbi.nlm.nih.gov/pubmed/32342614
35. Ding R, Medeiros M, Dadhania D, Muthukumar T, Kracker D, Kong J M, et al. Noninvasive diagnosis of BK virus nephritis by measurement of messenger RNA for BK virus VP1 in urine1. Transplantation [Internet]. 2002 Oct. 15 [cited 2019 Aug. 14]; 74(7):987-94. Available from: http://www.ncbi.nlm.nih.gov/pubmed/12394843
36. Loupy A, Haas M, Solez K, Racusen L, Glotz D, Seron D, et al. The Banff 2015 Kidney Meeting Report: Current Challenges in Rejection Classification and Prospects for Adopting Molecular Pathology. Am J Transplant [Internet]. 2017 Jan. [cited 2020 Mar. 17]; 17(1):28-41. Available from: http://www.ncbi.nlm.nih.gov/pubmed/27862883
37. Madden K, Janitell C, Sower D, Yang S. Prediction of BK viremia by urine viral load in renal transplant patients: An analysis of BK viral load results in paired urine and plasma samples. Transpl Infect Dis [Internet]. 2018 Oct.; 20(5):e12952. Available from: http://www.ncbi.nlm.nih.gov/pubmed/29896858

1. A method for calculating a probability to have a risk of an acute rejection in a kidney transplant recipient by using the following equation:
$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

2. The method according to claim 1 is suitable for diagnosing an acute rejection in a kidney transplant recipient by calculating the probability.

3. The method according to claim 1 comprising further the following steps:
i) calculating a probability to have a risk of an acute rejection for said recipient using the following equation:
$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} ,$$
ii) comparing this probability with a predetermined reference value; and
iii) concluding that the kidney transplant recipient is having or is susceptible to have a risk of an acute rejection when the probability is higher than the predetermined reference value or concluding that the kidney transplant recipient is not having or is not susceptible to have a risk of an acute rejection when the probability is lower than the predetermined reference value.

4. The method according to claim 1 wherein the probability (p) is determined with levels of two proteins expression in a biological sample obtained from a kidney transplant recipient and six clinical parameters of said recipient.

5. The method according to claim 1 wherein said two proteins are CXCL9 and CXCL10, and wherein the six clinical parameters are: recipient sex, recipient age, estimated glomerular filtration rate, donor-specific anti-HLA antibodies score, blood BKV viral load and urinary tract infection.

6. The method according to claim 1, wherein the kidney transplant recipient is under immunosuppressive treatment.

7. The method according to claim 1, wherein said kidney transplant recipient has further been grafted with the pancreas, and optionally a piece of duodenum, of the kidney donor.

8. A method for determining whether a renal biopsy is required or not in a kidney transplant recipient by calculating a probability of acute rejection for said recipient by using the following equation:
$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

9. A method for predicting the subsequent occurrence of an acute rejection in a kidney transplant recipient comprising a step of calculating a probability of acute rejection for said recipient by using the following equation:
$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

10. A method for predicting whether a kidney transplant recipient is at risk of graft loss comprising a step of calculating the probability of acute rejection for said recipient by using the following equation:
$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} .$$

11. A method for predicting the survival time of a kidney transplant recipient comprising the steps of:
calculating the probability of acute rejection for said recipient by using the following
equation:
$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} ,$$
ii) comparing the probability calculated at step i) with its predetermined reference value and
iii) concluding that the subject will have a short survival time when the probability is higher than its predetermined reference value or concluding that the subject will have a long survival time when the probability is lower than its predetermined reference value.

12. A method for preventing and/or treating acute rejection or progression of acute rejection in a kidney transplanted recipient, comprising the steps of: (i) performing the method for diagnosing acute rejection according to the method of claim 1 and (ii) administering to said recipient a therapeutically effective amount of a compound selected from the group consisting of azathioprine, tacrolimus, rapamycin derivative (sirolimus and everolimus), mycophenolic acid (mycophenolate mofetil and anteric-coated mycophenolate sodium), corticosteroids, and cyclosporins.

13. An immunosuppressive therapy for use in treating a kidney transplanted recipient, wherein said kidney transplant recipient subject is diagnosed as being at risk of having an acute rejection by the method according to claim 1.

14. A method for identifying a kidney recipient subject under immunosuppressive therapy as a candidate for immunosuppressive therapy weaning or minimization, comprising the steps of:
i) determining whether the subject is at risk of having an acute rejection by the method according to claim 1; and
ii) concluding that the kidney recipient subject is eligible to immunosuppressive therapy weaning or minimization when the subject is not at risk to have an acute rejection.

15. A kit for performing the method according to claim 1, wherein said kit comprises (i) means for determining the expression level of the CXL9 and CXCL10 in a biological sample obtained from said kidney transplant recipient and (ii) means for determining the six clinical parameters.

16. A method for diagnosing acute rejection in a kidney transplanted recipient, comprising the following steps:
i) calculating a probability to have a risk of an acute rejection for said recipient using the following equation:
$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} ,$$
ii) comparing this probability with a predetermined reference value; and
iii) concluding that the kidney transplant recipient is having or is susceptible to have a risk of an acute rejection when the probability is higher than the predetermined reference value or concluding that the kidney transplant recipient is not having or is not susceptible to have a risk of an acute rejection when the probability is lower than the predetermined reference value, wherein:
(x1) is recipient sex,
(x2) is recipient age,
(x3) is estimated glomerular filtration rate,
(x4) is donor-specific anti-HLA antibodies score,
(x5) is blood BKV viral load,
(x6) is urinary tract infection,
(x7) is CXCL9 and
(x8) is CXCL10.

17. A computer-implemented method for diagnosing acute rejection in a kidney transplanted recipient, comprising the following steps:
i) calculating a probability to have a risk of an acute rejection for said recipient using the following equation:
$$p = \frac{1}{\begin{matrix}1 + exp ( - ( β0 + β1 x 1 + β2 x 2 + β3 x 3 + β4 x 4 + \\ β5 x 5 + β6 x 6 + β7 x 7 + β8 x 8 ) )\end{matrix}} ,$$
ii) comparing this probability with a predetermined reference value; and
iii) concluding that the kidney transplant recipient is having or is susceptible to have a risk of an acute rejection when the probability is higher than the predetermined reference value or concluding that the kidney transplant recipient is not having or is not susceptible to have a risk of an acute rejection when the probability is lower than the predetermined reference value, wherein:
(x1) is recipient sex,
(x2) is recipient age,
(x3) is estimated glomerular filtration rate,
(x4) is donor-specific anti-HLA antibodies score,
(x5) is blood BKV viral load,
(x6) is urinary tract infection,
(x7) is CXCL9 and
(x8) is CXCL10.

18. A computer program product comprising code instructions for implementing the method according to claim 1, when it is executed by a computer.
